SEQUENCE LISTING (18 sequences)

SEQ ID NO: 1 - DNA, 10091 bp, Artificial Sequence
  Synthetic Sequence
aatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgctagcttttaaaagaaaaggggggattggggggtacagtgcaggggaaagaatagtagacataatagcaacagacatacaaactaaagaattacaaaaacaaattacaaaaattcaaaattttactagtgattatcggatcaactttgtatagaaaagttgggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggtctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacaagtttgtacaaaaaagcaggctgccaccatggaaagctctgccaagatggagagcggcggcgccggccagcagccccagccgcagccccagcagcccttcctgccgcccgcagcctgtttctttgccacggccgcagccgcggcggccgcagccgccgcagcggcagcgcagagcgcgcagcagcagcagcagcagcagcagcagcagcagcaggcgccgcagctgagaccggcggccgacggccagccctcagggggcggtcacaagtcagcgcccaagcaagtcaagcgacagcgctcgtcttcgcccgaactgatgcgctgcaaacgccggctcaacttcagcggctttggctacagcctgccgcagcagcagccggccgccgtggcgcgccgcaacgagcgcgagcgcaaccgcgtcaagttggtcaacctgggctttgccacccttcgggagcacgtccccaacggcgcggccaacaagaagatgagtaaggtggagacactgcgctcggcggtcgagtacatccgcgcgctgcagcagctgctggacgagcatgacgcggtgagcgccgccttccaggcaggcgtcctgtcgcccaccatctcccccaactactccaacgacttgaactccatggccggctcgccggtctcatcctactcgtcggacgagggctcttacgacccgctcagccccgaggagcaggagcttctcgacttcaccaactggttctgaacagtgacccagctttcttgtacaaagtggtgataatcgaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcccgcggttcgaacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagggctccggagagggcaggggaagtcttctaacatgcggggacgtggaggaaaatcccggccccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgaggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagagagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagctt

SEQ ID NO: 2 - DNA, 58 bp, Artificial Sequence
  Synthetic Sequence
  misc_feature (5)..(25): n is a, c, g, or t
  misc_feature (32)..(52): n is a, c, g, or t
ccggnnnnnnnnnnnnnnnnnnnnnctcgagnnnnnnnnnnnnnnnnnnnnntttttg

SEQ ID NO: 3 - DNA, 229 bp, Artificial Sequence
  Synthetic sequence
aatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaac

SEQ ID NO: 4 - DNA, 181 bp, Artificial Sequence
  Synthetic sequence
gggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagca

SEQ ID NO: 5 - DNA, 45 bp, Artificial Sequence
  Synthetic sequence
tgagtacgccaaaaattttgactagcggaggctagaaggagagag

SEQ ID NO: 6 - DNA, 234 bp, Artificial Sequence
  Synthetic sequence
aggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcct

SEQ ID NO: 7 - DNA, 118 bp, Artificial Sequence
  Synthetic Sequence
ttttaaaagaaaaggggggattggggggtacagtgcaggggaaagaatagtagacataatagcaacagacatacaaactaaagaattacaaaaacaaattacaaaaattcaaaatttt

SEQ ID NO: 8 - DNA, 1179 bp, Artificial Sequence
  Synthetic Sequence
ggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggtctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 9 - DNA, 6 bp, Artificial Sequence
  Synthetic Sequence
gccacc

SEQ ID NO: 10 - DNA, 711 bp, Artificial Sequence
  Synthetic Sequence
atggaaagctctgccaagatggagagcggcggcgccggccagcagccccagccgcagccccagcagcccttcctgccgcccgcagcctgtttctttgccacggccgcagccgcggcggccgcagccgccgcagcggcagcgcagagcgcgcagcagcagcagcagcagcagcagcagcagcagcaggcgccgcagctgagaccggcggccgacggccagccctcagggggcggtcacaagtcagcgcccaagcaagtcaagcgacagcgctcgtcttcgcccgaactgatgcgctgcaaacgccggctcaacttcagcggctttggctacagcctgccgcagcagcagccggccgccgtggcgcgccgcaacgagcgcgagcgcaaccgcgtcaagttggtcaacctgggctttgccacccttcgggagcacgtccccaacggcgcggccaacaagaagatgagtaaggtggagacactgcgctcggcggtcgagtacatccgcgcgctgcagcagctgctggacgagcatgacgcggtgagcgccgccttccaggcaggcgtcctgtcgcccaccatctcccccaactactccaacgacttgaactccatggccggctcgccggtctcatcctactcgtcggacgagggctcttacgacccgctcagccccgaggagcaggagcttctcgacttcaccaactggttctga

SEQ ID NO: 11 - DNA, 6 bp, Artificial Sequence
  Synthetic Sequence
acagtg

SEQ ID NO: 12 - DNA, 598 bp, Artificial Sequence
  Synthetic Sequence
cgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgg

SEQ ID NO: 13 - DNA, 588 bp, Artificial Sequence
  Synthetic sequence
gttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctc

SEQ ID NO: 14 - DNA, 1380 bp, Artificial Sequence
  Synthetic sequence
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagggctccggagagggcaggggaagtcttctaacatgcggggacgtggaggaaaatcccggccccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctga

SEQ ID NO: 15 - DNA, 235 bp, Artificial Sequence
  Synthetic Sequence
ctggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagca

SEQ ID NO: 16 - DNA, 135 bp, Artificial Sequence
  Synthetic sequence
acttgtttattgcagcttataatggttacaaataaagcaatagcatcacaaatttcacaaataaagcatttttttcactgcattctagttgtggtttgtccaaactcatcaatgtatcttatcatgtctggctct

SEQ ID NO: 17 - DNA, 861 bp, Artificial Sequence
  Synthetic sequence
atgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaa

SEQ ID NO: 18 - DNA, 589 bp, Artificial Sequence
  Synthetic sequence
ttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagagagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaa

(19) United States
(12) Patent Application Publication
(10) Pub. No.: US 2022/0056520 A1
(43) Pub. Date: Feb. 24, 2022

(54) METHODS FOR HIGH-THROUGHPUT SCREENING FOR GENES RELATING TO CELLULAR DIFFERENTIATION

(71) Applicant: The Research Foundation for the State University of New York, Albany, NY (US)

(72) Inventor: Chunyu Liu, Manlius, NY (US)

(21) Appl. No.: 17/357,915

(22) Filed: Jun. 24, 2021

Related U.S. Application Data
(60) Provisional application No. 63/043,602, filed on Jun. 24, 2020.

(51) Int. Cl. C12Q 1/6874; C12N 15/86; C12N 5/079

(52) U.S. Cl. CPC C12Q 1/6874; C12N 15/86; C12N 2506/02; C12N 2740/15043; C12N 2510/00; C12N 5/0618

(57) ABSTRACT

A method of identifying genes relating to cellular differentiation is provided herein. In some embodiments, a method of identifying regulatory genes relating to cellular differentiation includes: contacting a plurality of stem cells with one or more tagged regulatory genes and a selection marker to form a first plurality of transfected/transduced stem cells; selecting the first plurality of transfected/transduced stem cells; culturing the plurality of transfected/transduced stem cells under conditions suitable to allow the plurality of transfected/transduced stem cells to differentiate into a plurality of differentiated cells expressing the one or more tagged regulatory genes; and performing a single cell RNA sequencing on the plurality of differentiated cells to identify genes relating to cellular differentiation.

### CROSS-REFERENCE TO RELATED APPLICATIONS

The present disclosure claims priority or the benefit under 35 U.S.C. § 119 of U.S. provisional application No. 63/043,602 filed Jun. 24, 2020, the contents of which are fully incorporated herein by reference.

### REFERENCE TO A SEQUENCE LISTING

This application contains a Sequence Listing in computer readable form, which is incorporated herein by reference.

### FIELD OF THE INVENTION

The present disclosure relates generally to the field of cell biology. More specifically to methods for identifying one or more genes relating to cellular differentiation, and culture conditions and materials that facilitate differentiation and use of stem cells.

### BACKGROUND

Stem cells are cells that can divide without limit and develop into specialized cell types. Stem cells may be Adult Stem Cells (ASC), Embryonic Stem Cells (ESC), or Induced Pluripotent Stem cells (iPSC). ASC are undifferentiated cells found within tissues, which can renew themselves, and replenish damaged or dead tissues. ESC are found within an embryo, these cells are pluripotent and have the ability to differentiate into almost any specialized terminal cell type. iPSC are cells created in a laboratory wherein an embryonic gene is introduced into a somatic cell, which reverts the cell back into a stem cell-like state. Similar, to ESC, iPSC are able to differentiate into specialized terminal cell types.

Specialized terminal differentiated cells that begin from a common stem cell all have the same DNA expressed within the cell, even though they are expressing different genes. These specialized terminal cells arise through cellular differentiation as the cell focuses on a certain regulatory gene within the DNA. However, the inventor has found that mechanisms and genes which induce the stem cells to differentiate into specialized terminal cells are not well understood.

One of the many draws of stem cell research is the potential uses in regenerative medicine. Utilizing stem cells there is a potential to regenerate tissues, nerves, and similar organs from the donor/recipient, instead of the patient having to undergo a transplant. However, in order to utilize the stem cells in this way the ability to predict and control cellular differentiation is necessary. Predictability and control result from knowing which regulatory genes lead to each type of specialized terminal cell, and these genes are currently hard to determine, and in practice are determined by chance.

Differentiation of stem cells into specific terminal cell types is an important life process, which is highly regulated by genes. Defects of such regulatory genes lead to various diseases. Unfortunately, many of such genes remain unknown, and there is no efficient method to identify such genes.

Prior art of interest includes US Patent Publication No. 2010/0239539 entitled Methods for promoting differentiation and differentiation efficiency (herein incorporated by reference). However, the methods discussed therein do not identify one or more genes relating to cellular differentiation or provide culture conditions and materials that facilitate differentiation and use of stem cells such as when identifying genes-of-interest.

Accordingly, there is a need for improved methods, apparatuses, and assays for the detection and identification of one or more regulatory genes required to induce a stem cell into cellular differentiation resulting in a specific specialized terminal cell, and the efficacy of each gene.

### SUMMARY

The present disclosure relates to methods for high-throughput screening for genes such as regulatory genes related to cell differentiation. In embodiments, a method of identifying genes relating to cellular differentiation is provided, the method including: contacting a plurality of stem cells with one or more tagged regulatory genes and a selection marker to form a first plurality of transfected/transduced stem cells; selecting the first plurality of transfected/transduced stem cells; culturing the first plurality of transfected/transduced stem cells under conditions suitable to allow the first plurality of transfected/transduced stem cells to differentiate into a plurality of differentiated cells expressing the one or more tagged regulatory genes; and performing a single cell RNA sequencing on the plurality of differentiated cells to identify genes relating to cellular differentiation.

In some embodiments, a method for identifying a regulatory gene relating to cellular differentiation includes: transfecting or transducing a plurality of stem cells within a cell culturing system with a test gene; incubating the cell culturing system under conditions suitable to allow the one or more stem cells including the test gene to differentiate into a plurality of differentiated cells; and performing single cell RNA sequencing on the plurality of differentiated cells, wherein the single cell RNA sequencing of the plurality of differentiated cells is indicative of the test gene's efficacy as a regulatory gene for cellular differentiation.

In some embodiments, the present disclosure relates to a non-transitory computer readable medium having instructions stored thereon that, when executed, causes an apparatus to perform a method, including: contacting a plurality of stem cells with one or more tagged regulatory genes and a selection marker to form a first plurality of transfected/transduced stem cells; selecting the first plurality of transfected/transduced stem cells; culturing the first plurality of transfected/transduced stem cells under conditions suitable to allow the plurality of transfected/transduced stem cells to differentiate into a plurality of differentiated cells expressing the one or more tagged regulatory genes; and performing a single cell RNA sequencing on the plurality of differentiated cells to identify genes relating to cellular differentiation.

In embodiments, the present disclosure relates to one or more DNA constructs including a promoter upstream a predetermined shRNA, which is upstream a gene-of-interest, which is upstream a barcode sequence. In embodiments, the DNA constructs are transduced/transfected into a cell such as a host cell. In embodiments, the DNA construct is either transduced into a cell, or transfected into a cell, but not both.

In embodiments, the present disclosure includes a first design including shRNA to knockdown a target gene. A second embodiments, overexpressed the one or more target genes.

The illustrative aspects of the present disclosure are designed to solve the problems herein described and/or other problems not discussed.

### BRIEF DESCRIPTION OF THE DRAWINGS AND SEQUENCES

Embodiments of the present disclosure, briefly summarized above and discussed in greater detail below, can be understood by reference to the illustrative embodiments of the disclosure depicted in the appended drawings. However, the appended drawings illustrate only typical embodiments of the disclosure and are therefore not to be considered limiting of scope, for the disclosure may admit to other equally effective embodiments.

FIG. 1 depicts a flow diagram of a method for identifying genes relating to cellular differentiation in accordance with the present disclosure.

FIG. 2 depicts a flow diagram of a method for identifying the efficacy of genes as a regulatory gene for cell differentiation in accordance with the present disclosure.

FIG. 3 depicts a flow diagram of one or more method for identifying genes relating to cellular differentiation in accordance with the present disclosure.

FIGS. 4A and 4B depict the expression dynamics of candidate genes in iPSC-derived cells. FIG. 4C depicts the expression profiles of the 20 selected genes in the transcriptome changes when iPSCs differentiate to neurons.

FIG. 5 depicts coding and decoding of genes that can induce stem cell differentiation.

FIG. 6 depicts overexpression lentivirus construction for the transfer plasmid.

FIGS. 7A and 7B depicts a lentivirus construct for shRNA knockdown screening in accordance with the present disclosure.

FIG. 8 depicts a vector suitable for use in accordance with the present disclosure.

SEQ ID NO: 1 depicts the sequence for an expression vector suitable for use in accidence with the present disclosure.

SEQ ID NO: 2 depicts the sequence for a lentivirus construct for shRNA knockdown screening in accordance with the present disclosure.

SEQ ID NOS: 3-18 are further described in Table 1 below.

It is noted that the drawings of the disclosure are not necessarily to scale. The drawings are intended to depict only typical aspects of the disclosure, and therefore should not be considered as limiting the scope of the disclosure. In the drawings, like numbering represents like elements between the drawings.

### DETAILED DESCRIPTION

Embodiments of the present disclosure provide methods for identifying regulatory genes relating to cellular differentiation. More specifically, the methods of the present disclosure provide ways to determine one or more regulatory genes required to induce a stem cell into cellular differentiation resulting in a specific specialized terminal cell, and the efficacy of each of the one or more identified genes such as regulatory genes. For example, embodiments include a method of identifying genes relating to cellular differentiation, the method including: contacting a plurality of stem cells with one or more tagged regulatory genes and a selection marker to form a first plurality of transfected or transduced stem cells; selecting the first plurality of transfected/transduced stem cells; culturing the plurality of transfected or transduced stem cells under conditions suitable to allow the plurality of transfected or transduced stem cells to differentiate into a plurality of differentiated cells expressing the one or more tagged regulatory genes; and performing a single cell RNA sequencing on the plurality of differentiated cells to identify genes relating to cellular differentiation. Advantages of the methods of the present disclosure include: the ability to simultaneously study multiple genes and/or combinations of genes; the ability to simultaneously determine each gene's efficacy as a regulatory gene; and providing an increased throughput for determining the efficacy of the genes.

### Definitions

As used in the present specification, the following words and phrases are generally intended to have the meanings as set forth below, except to the extent that the context in which they are used indicates otherwise.

As used herein, the singular forms “a”, “an”, and “the” include plural references unless the context clearly dictates otherwise. Thus, for example, references to “a compound” include the use of one or more compound(s). “A step” of a method means at least one step, and it could be one, two, three, four, five or even more method steps.

As used herein the terms “about,” “approximately,” and the like, when used in connection with a numerical variable, generally refers to the value of the variable and to all values of the variable that are within the experimental error (e.g., within the 95% confidence interval [CI 95%] for the mean) or within ±10% of the indicated value, whichever is greater.

As used herein the term “barcode,” generally refers to a label that may be attached to an analyte to convey information about the analyte. For example, a barcode may be a polynucleotide sequence attached to fragments of a target polynucleotide. This barcode may then be sequenced with the fragments of the target polynucleotide. In embodiments, the presence of the same barcode on multiple sequences may provide information about the origin of the sequence. For example, a barcode may indicate that the sequence came from a particular proximal region of a genome, a specific transgene vector. This may be particularly useful for sequence assembly when several nucleic acid constructs are pooled for inducing cell differentiation before sequencing.

As used herein the term “cDNA” refers to a DNA molecule that can be prepared by reverse transcription from an RNA molecule obtained from a eukaryotic or prokaryotic cell, a virus, or from a sample solution. In embodiments, cDNA lacks introns or intron sequences that may be present in corresponding genomic DNA. In embodiments, cDNA may refer to a nucleotide sequence that corresponds to the nucleotide sequence of an RNA from which it is derived. In embodiments, cDNA refers to a double-stranded DNA that is complementary to and derived from mRNA.

As used herein the term “coding sequence” means a polynucleotide, which directly specifies the amino acid sequence of a polypeptide. In embodiments, boundaries of the coding sequence may be determined by an open reading frame, which begins with a start codon such as ATG, GTG, or TTG and ends with a stop codon such as TAA, TAG, or TGA. The coding sequence may be a genomic DNA, cDNA, synthetic DNA, or a combination thereof.

The terms “deoxyribonucleotide” and “DNA” refer to a nucleotide or polynucleotide including at least one ribosyl moiety that has an H at the 2′ position of a ribosyl moiety. In embodiments, a deoxyribonucleotide is a nucleotide having an H at its 2′ position.

As used herein, the term “differentiation” means the process by which cells become progressively more specialized.

As used herein, the term “differentiation efficiency” means the percentage of cells in a population that are differentiating or are able to differentiate or the speed of cells differentiate.

As used herein, “conditioned medium” is a medium in which a specific cell or population of cells has been cultured, and then removed. In embodiments, when cells are cultured in a medium, they may secrete cellular factors that can provide support to or affect the behavior of other cells. Such factors include, but are not limited to hormones, cytokines, extracellular matrix (ECM), proteins, vesicles, antibodies, chemokines, receptors, inhibitors and granules. The medium containing the cellular factors is the conditioned medium. Examples of methods of preparing conditioned media are described in U.S. Pat. No. 6,372,494 which is incorporated by reference in its entirety herein. As used herein, conditioned medium also refers to components, such as proteins, that are recovered and/or purified from conditioned medium or from AMP cells.

By “hybridizable” or “complementary” or “substantially complementary” a nucleic acid (e.g. RNA, DNA) includes a sequence of nucleotides that enables it to non-covalently bind, i.e. form Watson-Crick base pairs and/or G/U base pairs, “anneal”, or “hybridize,” to another nucleic acid in a sequence-specific, antiparallel, manner (i.e., a nucleic acid specifically binds to a complementary nucleic acid) under the appropriate in vitro and/or in vivo conditions of temperature and solution ionic strength. Standard Watson-Crick base-pairing includes: adenine/adenosine) (A) pairing with thymidine/thymidine (T), A pairing with uracil/uridine (U), and guanine/guanosine) (G) pairing with cytosine/cytidine (C). In addition, for hybridization between two RNA molecules (e.g., dsRNA), and for hybridization of a DNA molecule with an RNA molecule (e.g., when a DNA target nucleic acid base pairs with a guide RNA, etc.): G can also base pair with U. For example, G/U base-pairing is partially responsible for the degeneracy (i.e., redundancy) of the genetic code in the context of tRNA anti-codon base-pairing with codons in mRNA. In embodiments, hybridization requires that the two nucleic acids contain complementary sequences, although mismatches between bases are possible. The conditions appropriate for hybridization between two nucleic acids depend on the length of the nucleic acids and the degree of complementarity, variables well known in the art. The greater the degree of complementarity between two nucleotide sequences, the greater the value of the melting temperature (Tm) for hybrids of nucleic acids having those sequences. Typically, the length for a hybridizable nucleic acid is 8 nucleotides or more (e.g., 10 nucleotides or more, 12 nucleotides or more, 15 nucleotides or more, 20 nucleotides or more, 22 nucleotides or more, 25 nucleotides or more, or 30 nucleotides or more). It is understood that the sequence of a polynucleotide need not be 100% complementary to that of its target nucleic acid to be specifically hybridizable. Moreover, a polynucleotide may hybridize over one or more segments such that intervening or adjacent segments are not involved in the hybridization event (e.g., a loop structure or hairpin structure, a ‘bulge’, and the like). A polynucleotide can include 60% or more, 65% or more, 70% or more, 75% or more, 80% or more, 85% or more, 90% or more, 95% or more, 98% or more, 99% or more, 99.5% or more, or 100% sequence complementarity to a target region within the target nucleic acid sequence to which it will hybridize. For example, an antisense nucleic acid in which 18 of 20 nucleotides of the antisense compound are complementary to a target region, and would therefore specifically hybridize, would represent 90 percent complementarity. The remaining noncomplementary nucleotides may be clustered or interspersed with complementary nucleotides and need not be contiguous to each other or to complementary nucleotides. Percent complementarity between particular stretches of nucleic acid sequences within nucleic acids can be determined using any convenient method. Example methods include BLAST programs (basic local alignment search tools) and PowerBLAST programs (Altschul et al., J. Mol. Biol., 1990, 215, 403-410; Zhang and Madden, Genome Res., 1997, 7, 649-656) or by using the Gap program (Wisconsin Sequence Analysis Package, Version 8 for Unix, Genetics Computer Group, University Research Park, Madison Wis.), e.g., using default settings, which uses the algorithm of Smith and Waterman (Adv. Appl. Math., 1981, 2, 482-489).

As used herein, “enriched” means to selectively concentrate or to increase the amount of one or more materials by elimination of the unwanted materials or selection and separation of desirable materials from a mixture (i.e. separate cells with specific cell markers from a heterogeneous cell population in which not all cells in the population express the marker).

As defined herein, a “gene” is the segment of DNA involved in producing a polypeptide chain; it includes regions preceding and following the coding region, as well as intervening sequences (introns) between individual coding segments (exons).

As used herein, a “regulatory gene” is a gene that regulates the expression of one or more structural genes by controlling the production of a protein (such as a genetic repressor) which regulates their rate of transcription.

As used herein, a “structural gene” is a gene encoding for the production of a specific RNA, structural protein, or enzyme not involved in regulation.

The term “isolated” means a substance in a form or environment that does not occur in nature. Non-limiting examples of isolated substances include (1) any non-naturally occurring substance, (2) any substance such as a variant, nucleic acid, protein, peptide or cofactor, that is at least partially removed from one or more or all of the naturally occurring constituents with which it is associated in nature; (3) any substance modified by the hand of man relative to that substance found in nature; or (4) any substance modified by increasing the amount of the substance relative to other components with which it is naturally associated.

The term “nucleotide” refers to a ribonucleotide or a deoxyribonucleotide or modified form thereof, as well as an analog thereof.

As used herein, the term “nucleic acid molecule” refers to any molecule containing multiple nucleotides (i.e., molecules comprising a sugar (e.g., ribose or deoxyribose) linked to a phosphate group and to an exchangeable organic base, which is either a substituted pyrimidine (e.g., cytosine (C), thymine (T) or uracil (U)) or a substituted purine (e.g., adenine (A) or guanine (G)). As described further below, bases include C, T, U, C, and G, as well as variants thereof. As used herein, the term refers to ribonucleotides (including oligoribonucleotides (ORN)) as well as deoxyribonucleotides (including oligodeoxynucleotides (ODN)). The term shall also include polynucleosides (i.e., a polynucleotide minus the phosphate) and any other organic base containing polymer. Nucleic acid molecules can be obtained from existing nucleic acid sources (e.g., genomic or cDNA), but include synthetic (e.g., produced by oligonucleotide synthesis). In embodiments, the terms “nucleic acid” “nucleic acid molecule” and “polynucleotide” may be used interchangeably herein, and refer to both RNA and DNA, including cDNA, genomic DNA, synthetic DNA, and DNA (or RNA) containing nucleic acid analogs. Polynucleotides can have any three-dimensional structure. A nucleic acid can be double-stranded or single-stranded (i.e., a sense strand or an antisense strand). Non-limiting examples of polynucleotides include genes, gene fragments, exons, introns, messenger RNA (mRNA) and portions thereof, transfer RNA, ribosomal RNA, siRNA, micro-RNA, ribozymes, cDNA, recombinant polynucleotides, branched polynucleotides, plasmids, vectors, isolated DNA of any sequence, isolated RNA of any sequence, nucleic acid probes, and primers, as well as nucleic acid analogs.

In embodiments, the term “oligonucleotide” refers to a polynucleotide of between 4 and 100 nucleotides of single- or double-stranded nucleic acid (e.g., DNA, RNA, or a modified nucleic acid). However, for the purposes of this disclosure, there is no upper limit to the length of an oligonucleotide. Oligonucleotides are also known as “oligomers” or “oligos” and can be isolated from genes, transcribed (in vitro and/or in vivo), or chemically synthesized.

The terms “peptide,” “polypeptide,” and “protein” are used interchangeably herein, and refer to a polymeric form of amino acids of any length, which can include coded and non-coded amino acids, chemically or biochemically modified or derivatized amino acids, and polypeptides having modified peptide backbones.

The terms “polynucleotide” and “nucleic acid,” used interchangeably herein, refer to a polymeric form of nucleotides of any length, either ribonucleotides or deoxyribonucleotides. Thus, terms “polynucleotide” and “nucleic acid” encompass single-stranded DNA; double-stranded DNA; multi-stranded DNA; single-stranded RNA; double-stranded RNA; multi-stranded RNA; genomic DNA; cDNA; DNA-RNA hybrids; and a polymer including purine and pyrimidine bases or other natural, chemically or biochemically modified, non-natural, or derivatized nucleotide bases. The terms “polynucleotide” and “nucleic acid” should be understood to include, as applicable to the embodiments being described, single-stranded (such as sense or antisense) and double-stranded polynucleotides.

As used herein, the term “protein marker” means any protein molecule characteristic of a cell or cell population. The protein marker may be located on the plasma membrane of a cell or in some cases may be a secreted protein.

The terms “sequence identity”, “identity” and the like as used herein with respect to polynucleotide or polypeptide sequences refer to the nucleic acid residues or amino acid residues in two sequences that are the same when aligned for maximum correspondence over a specified comparison window. Thus, “percentage of sequence identity”, “percent identity” and the like refer to the value determined by comparing two optimally aligned sequences over a comparison window, wherein the portion of the polynucleotide or polypeptide sequence in the comparison window may include additions or deletions (i.e., gaps) as compared to the reference sequence (which does not comprise additions or deletions) for optimal alignment of the two sequences. The percentage may be calculated by determining the number of positions at which the identical nucleic acid base or amino acid residue occurs in both sequences to yield the number of matched positions, dividing the number of matched positions by the total number of positions in the window of comparison and multiplying the results by 100 to yield the percentage of sequence identity.

It would be understood that, when calculating sequence identity between a DNA sequence and an RNA sequence, T residues of the DNA sequence align with, and can be considered “identical” with, U residues of the RNA sequence. For purposes of determining “percent complementarity” of first and second polynucleotides, one can obtain this by determining (i) the percent identity between the first polynucleotide and the complement sequence of the second polynucleotide (or vice versa), for example, and/or (ii) the percentage of bases between the first and second polynucleotides that would create canonical Watson and Crick base pairs. In embodiments, the degree of sequence identity between a query sequence and a reference sequence is determined by: 1) aligning the two sequences by any suitable alignment program using the default scoring matrix and default gap penalty; 2) identifying the number of exact matches, where an exact match is where the alignment program has identified an identical amino acid or nucleotide in the two aligned sequences on a given position in the alignment; and 3) dividing the number of exact matches with the length of the reference sequence. In one embodiment, the degree of sequence identity between a query sequence and a reference sequence is determined by: 1) aligning the two sequences by any suitable alignment program using the default scoring matrix and default gap penalty; 2) identifying the number of exact matches, where an exact match is where the alignment program has identified an identical amino acid; or nucleotide in the two aligned sequences on a given position in the alignment; and 3) dividing the number of exact matches with the length of the longest of the two sequences. In some embodiments, the degree of sequence identity refers to and may be calculated as described under “Degree of Identity” in U.S. Pat. No. 10,531,672 starting at Column 11, line 56. U.S. Pat. No. 10,531,672 is incorporated by reference in its entirety. In embodiments, an alignment program suitable for calculating percent identity performs a global alignment program, which optimizes the alignment over the full-length of the sequences. In embodiments, the global alignment program is based on the Needleman-Wunsch algorithm (Needleman, Saul B.; and Wunsch, Christian D. (1970), “A general method applicable to the search for similarities in the amino acid sequence of two proteins”, Journal of Molecular Biology 48 (3): 443-53). Examples of current programs performing global alignments using the Needleman-Wunsch algorithm are EMBOSS Needle and EMBOSS Stretcher programs, which are both available on the world wide web at www.ebi.ac.uk/Tools/psa/. In some embodiments a global alignment program uses the Needleman-Wunsch algorithm and the sequence identity is calculated by identifying the number of exact matches identified by the program divided by the “alignment length”, where the alignment length is the length of the entire alignment including gaps and overhanging parts of the sequences. In embodiments, the mafft alignment program is suitable for use herein.

The term “substantially purified,” as used herein, refers to a component of interest that may be substantially or essentially free of other components which normally accompany or interact with the component of interest prior to purification. By way of example only, a component of interest may be “substantially purified” when the preparation of the component of interest contains less than about 30%, less than about 25%, less than about 20%, less than about 15%, less than about 10%, less than about 5%, less than about 4%, less than about 3%, less than about 2%, or less than about 1 (by dry weight) of contaminating components. Thus, a “substantially purified” component of interest may have a purity level of about 70%, about 75%, about 80%, about 85%, about 90%, about 95%, about 96%, about 97%, about 98%, about 99% or greater.

“Substantially similar” refers to nucleic acid molecules wherein changes in one or more nucleotide bases result in substitution of one or more amino acids, but do not affect the functional properties of the protein encoded by the DNA sequence. “Substantially similar” also refers to nucleic acid molecules wherein changes in one or more nucleotide bases do not affect the ability of the nucleic acid molecule to mediate alteration of gene expression by antisense or co-suppression technology. “Substantially similar” also refers to modifications of the nucleic acid molecules of the instant disclosure (such as deletion or insertion of one or more nucleotide bases) that do not substantially affect the functional properties of the resulting transcript vis-a-vis the ability to mediate alteration of gene expression by antisense or co-suppression technology or alteration of the functional properties of the resulting protein molecule. The disclosure encompasses more than the specific exemplary sequences.

As used herein, the term “target activity” refers to a biological activity capable of being modulated by a selective modulator. Certain exemplary target activities include, but are not limited to, binding affinity, signal transduction, enzymatic activity, tumor growth, inflammation or inflammation-related processes, and amelioration of one or more symptoms associated with a disease or condition.

As used herein, the term “target protein” refers to a molecule or a portion of a protein capable of being bound by a selective binding compound.

As used herein, the term “pluripotent stem cells” shall have the following meaning. Pluripotent stem cells are true stem cells with the potential to make any differentiated cell in the body, but cannot contribute to making the components of the extraembryonic membranes which are derived from the trophoblast. The amnion develops from the epiblast, not the trophoblast. Three types of pluripotent stem cells have been confirmed to date: Embryonic Stem (ES) Cells (may also be totipotent in primates), Embryonic Germ (EG) Cells, and Embryonic Carcinoma (EC) Cells. These EC cells can be isolated from teratocarcinomas, a tumor that occasionally occurs in the gonad of a fetus. Unlike the other two, they are usually aneuploid.

As used herein, the term “multipotent stem cells” are true stem cells but can only differentiate into a limited number of types. For example, the bone marrow contains multipotent stem cells that give rise to all the cells of the blood but may not be able to differentiate into other cells types.

As used herein, the term “hematopoietic stem cell” or “HSC” means a stem cell that is capable of differentiating into both myeloid lineages (i.e. monocytes, macrophages, neutrophils, basophils, eosinophils, erythrocytes, megakaryocytes/platelets and some dendritic cells) and lymphoid lineages (i.e. T-cells, B-cells, NK-cells, and some dendritic cells).

As used herein a “terminal cell” or “terminally differentiated cell” are synonymous and refer to cells that do not transform into other types of cells.

As used herein, the term “transcription” refers to a process of constructing a messenger RNA molecule using a DNA molecule as a template with resulting transfer of genetic information to the messenger RNA.

As used herein “transfection” or “transfected” refers to introducing naked or purified nucleic acids into eukaryotic cells by non-viral methods.

As used herein, “transduced” or “transduction” refers to a process of virus-mediated nucleic acid or gene transfer into eukaryotic cells.

General methods in molecular and cellular biochemistry can be found in such standard textbooks as Molecular Cloning: A Laboratory Manual, 3rd Ed. (Sambrook et al., HaRBor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference. In embodiments, there may be employed conventional molecular biology, microbiology, and recombinant DNA techniques within the skill of the art. Such techniques are explained fully in the literature. See, e.g., Sambrook et al, 2001, “Molecular Cloning: A Laboratory Manual”; Ausubel, ed., 1994, “Current Protocols in Molecular Biology” Volumes I-III; Celis, ed., 1994, “Cell Biology: A Laboratory Handbook” Volumes I-III; Coligan, ed., 1994, “Current Protocols in Immunology” Volumes I-III; Gaited., 1984, “Oligonucleotide Synthesis”; Hames & Higgins eds., 1985, “Nucleic Acid Hybridization”; Hames & Higgins, eds., 1984, “Transcription And Translation”; Freshney, ed., 1986, “Animal Cell Culture”; IRL Press, 1986, “Immobilized Cells And Enzymes”; Perbal, 1984, “A Practical Guide To Molecular Cloning.”

Before embodiments are further described, it is to be understood that this disclosure is not limited to particular embodiments described, as such may, of course, vary. It is also to be understood that the terminology used herein is for the purpose of describing particular embodiments only and is not intended to be limiting.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

### DESCRIPTION OF CERTAIN EMBODIMENTS

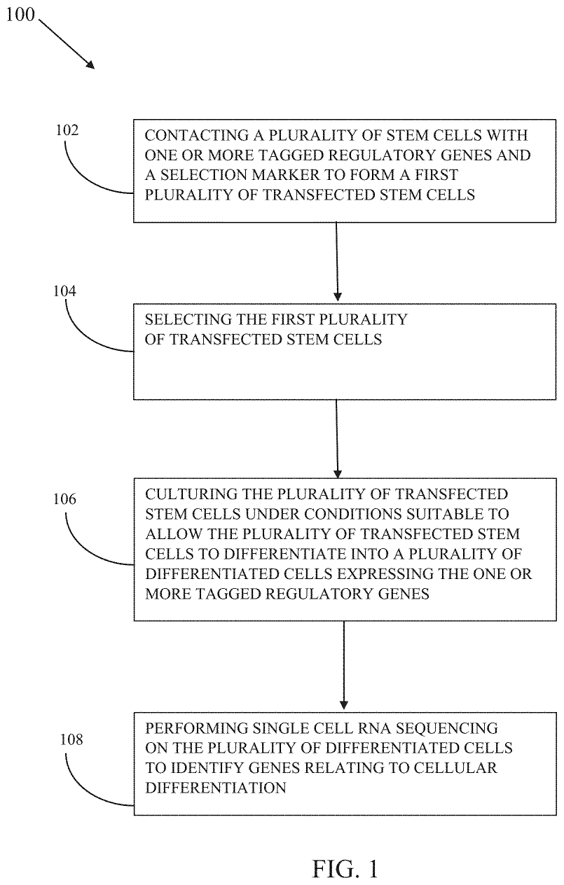

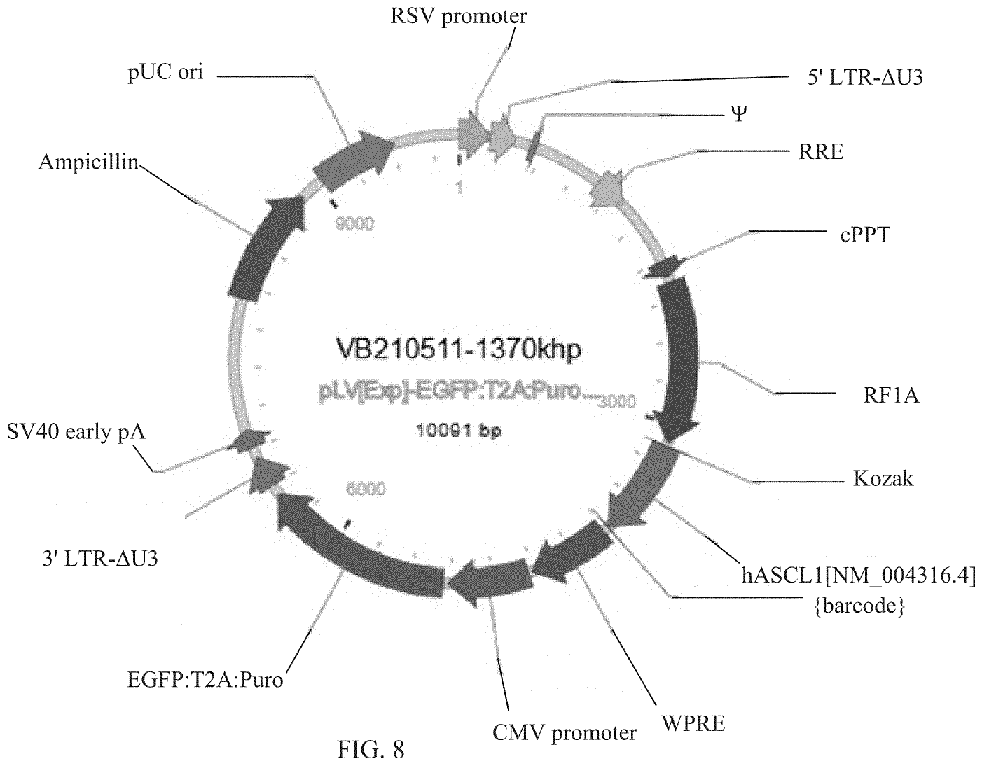

FIG. 1 is a flow diagram of a method 100 for identifying genes relating to cellular differentiation in accordance with some embodiments of the present disclosure. The method 100 includes at process sequence 102 contacting a plurality of stem cells with one or more tagged regulatory genes and a selection marker to form a first plurality of transfected stem cells. The plurality of stem cells can be prepared according to methods known in the art such as those described in Miskinyte et al., Direct Conversion of Human Fibroblasts to Functional Excitatory Cortical Neurons Integrating Into Human Neural Networks, Stem Cell Research & Therapy (2017) 8:207 (herein entirely incorporated by reference). See for example, the methods section including cell culture described therein. For example, in some embodiments a retrovirus construct carries one or more preselected tagged regulatory genes to a plurality of stem cells. In some embodiments the retrovirus construct is derived from a Lentivirus construct, but any acceptable retrovirus construct could be used. In embodiments, the Lentivirus construct includes one or more features of the nucleic acid construct depicted in FIG. 8. In embodiments, a suitable nucleic acid construct includes the nucleic acid construct of SEQ ID NOS: 1 or 2.

Further, in some embodiments, a retrovirus can deliver a selection marker to the plurality of stem cells. For example, in embodiments a non-limiting example of a selection marker includes an antibiotic marker, while in other embodiments, another selection marker known in the art may be used. In embodiments, an expression vector may include one or more genes for a preselected selective marker.

In embodiments, contacting a plurality of stem cells with one or more tagged regulatory genes and a selection marker to form a first plurality of transfected stem cells includes providing a plurality of stem cells. In embodiments, suitable stem cells for use herein include stem cells that are undifferentiated cells having an ability at the single cell level to both self-renew and differentiate to produce progeny cells, including self-renewing progenitors, non-renewing progenitors, and terminally differentiated cells. In embodiments, stem cells are also characterized by their ability to differentiate in vitro into functional cells of various ceil lineages from multiple germ layers (endoderm, mesoderm and ectoderm), as well as to give rise to tissues of multiple germ layers following transplantation and to contribute substantially to most, if not all, tissues following injection into blastocysts.

In embodiments, stem cells are often categorized on the basis of the source from which they may be obtained. In one embodiment, the neural progenitor cell preparation is produced from a population of embryonic stem cells. Embryonic stem cells are pluripotent cells that are derived from the inner cell mass of a blastocyst-stage embryo. In embodiments, these cell types may be provided in the form of an established cell line, or they may be obtained directly from primary embryonic tissue and used immediately for differentiation. Exemplary embryonic stem cells include those listed in the NIH Human Embryonic Stem Cell Registry, e.g. hESBGN-01, hESBGN-02, hESBGN-03, hESBGN-04 (BresaGen, Inc.).

In embodiments, stem cells may include Induced pluripotent stem cells. In embodiments, iPSCs may be derived by methods known in the art including the use integrating viral vectors (e.g., lentiviral vectors) to deliver the genes that promote cell reprogramming (See e.g., U.S. Patent Publication No. 20170321188, herein entirely incorporated by reference).

In embodiments, a population of stem cells, such as pluripotent stem cells, can be propagated continuously in culture, using culture conditions that promote proliferation without promoting differentiation. (See e.g., U.S. Patent Publication No. 20170321188 (herein entirely incorporated by reference).

In one embodiment of the present invention, a nucleic acid encoding one or more tagged regulatory genes and a selection marker or an expression vector comprising a nucleic acid molecule encoding one or more tagged regulatory genes and a selection marker is administered to a population of stem cells. The regulatory genes and selection marker may then be expressed from the nucleic acid molecule. In embodiments, suitable expression vectors include, viral vectors, such as lentiviral vectors.

In embodiments, the source of stem cells such as pluripotent stem cells, whether they are embryonic stem cells, fetal stem cells, iPSCs, etc., can be from any source, including mammalian sources, e.g., domesticated animals, such as cats and dogs; livestock (e.g., cattle, horses, pigs, sheep, and goats); laboratory animals (e.g., mice, rabbits, rats, and guinea pigs); non-human primates, and humans.

In embodiments, tagged regulatory genes may include a sequence including a base pair barcode. In embodiments, a base pair barcode for use herein includes a 4-10, or 5-10, or 6-10 base pair barcode, but any acceptable base pair barcode would be acceptable such as 4, 5, 6, 7, 8, 9, or 10 base pair barcode. In embodiments, the barcode is characterized as (n)₄₋₁₀, or (n)₅₋₁₀, wherein n is any nucleic acid. In some embodiments the base pair barcode is at a 5′ UTR or a 3′ UTR, where it will be transcribed and serve as an identifier in the transcriptome for the tagged regulatory genes, but not translated into protein. In some embodiments one or more tagged regulatory genes may include one or more genes found within the human genome. In further embodiments the tagged regulatory gene can be a coding gene, while in other embodiments the tagged regulatory gene can be a non-coding gene. Non-limiting examples of suitable regulatory genes include one or more of: ASCL1, PBRM1, RERE, CPEB1, ZSCAN2, ZNF536, PCBL11B, PBX4, ZNF491 SATB2, ARNT, GABPB2, SREBF1, SETDB1, NFATC3, ZNF440, TCF4, STAT6, TBX6, NR1H3, and others.

Still referring to FIG. 1, method 100 includes at process sequence 104 selecting a first plurality of transfected/transduced cells 104. In embodiments, the selection marker and selection technique are related to antibiotic markers, and antibiotics, however any sufficient marker, and selection agent may be appropriate. Such as fluorescent marker genes that can be used for cell sorting or for monitoring cell growth and differentiation, or other surface proteins that can be tagged by antibodies. In embodiments, selecting the first plurality of transfected/transduced cells may include contacting the stem cells with one or more antibiotics in an amount sufficient to kill the plurality of stem cells without the selection marker. In embodiments antibiotic suitable for use herein includes penicillin, cephalosporin, tetracyclines, aminoglycosides, quinolones, lincomycin, macrolides, sulfonamides, and glycopeptides. While in other embodiments any suitable antibiotic may be used.

Further the method 100 includes at process sequence 106 culturing the plurality of transfected/transduced stem cells under conditions suitable to allow the plurality of transfected/transduced stem cells to differentiate into a plurality of differentiated cells expressing the one or more tagged regulatory genes. The cells are then cultured for a period of time. In some embodiments the time can be 5-100 days, preferably 25-75 days, even more preferred is between 45 and 55 days. In some embodiments, the culturing is performed under conditions described in Miskinyte et al., Direct Conversion of Human Fibroblasts to Functional Excitatory Cortical Neurons Integrating Into Human Neural Networks, Stem Cell Research & Therapy (2017) 8:207. See e.g., the section described therein under co-culture of Ctx cells and adult human cortex organotypic slice cultures. In embodiments, during the culturing period the stem cells with the tagged regulatory genes can differentiate into subtype cells. In some embodiments the subtype cells can be excitatory, inhibitory neurons, astrocytes, oligodendrocyte or microglia, or any differentiated somatic cells. In embodiments, once the cells are differentiated the cells can be harvested. In embodiments, culturing conditions such as those known in the art may be used (See e.g., U.S. Patent Publication No. 20170321188 to Andrea Viczian (herein entirely incorporated by reference).

The method 100 further includes, at process sequence 108 performing single cell RNA sequencing on the differentiated cells to identify genes relating to cellular differentiation. Single cell RNA sequencing can be performed by methods described in Cuomo et al., Single-Cell RNA-sequencing of differentiating iPS cells reveals dynamic genetic effects on gene expression,” published Feb. 10, 2020 (herein entirely incorporated by reference). See e.g., the methods section therein including Pooled scRNA-seq profiling during endoderm differentiation, cell culture for maintenance and differentiation, single cell preparation and sorting for scRNA seq, immunofluorescence staining, fluorescence activated cell sorting (FACS) analysis, RNA isolation and RT-quantitative (q)PCR, genotyping, demultiplexing donors from pooled experiments, and scRNA-seq quality control and processing described therein. In embodiments, analyzing the RNA sequencing data involves grouping all cells expressing the same tagged regulatory genes based on barcodes as described above. Then the grouping of cells can be clustered using UMAP, t-SNE or similar methodology. In embodiments, each cluster of the cells can be classified into one or more subtypes based on the tagged genes which are expressed. Further, the tagged regulatory genes can be linked to the cell types identifying genes that drive the differentiation. In embodiments, the expression levels of the tagged regulatory genes are correlated with the cell proportion in the culture mix.

In embodiments, the method 100 can test many or a plurality of genes and their random combinations for their impact on cell differentiation and development. Further, in embodiments, RNA sequencing can be performed at different time points. In embodiments, the time variation may allow for quantifying the cell proportion to quantify the speed of the cell differentiation. The time points can range from hours to days, or weeks.

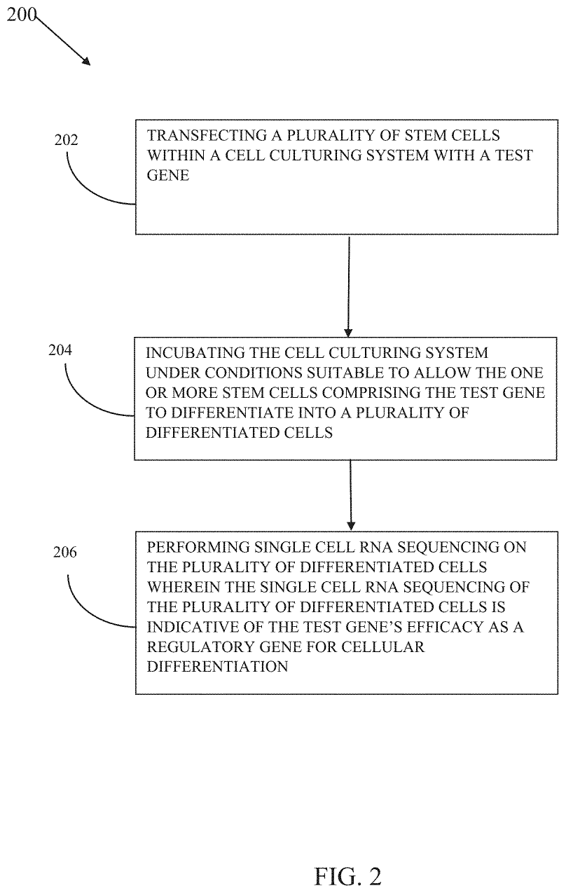

Referring now to FIG. 2 a flow diagram of a method for identifying the efficacy of genes as a regulatory gene for cell differentiation in accordance with the present disclosure is shown. In embodiments, the method 200 relates to identifying a regulatory gene relating to cellular differentiation. The method 200 includes at process sequence 202 transfecting/transducing a plurality of stem cells within a cell culturing system with a test gene. In some embodiments transfecting/transducing the stem cells can be achieved through tagging the test gene and introducing the gene to the stem cell culture through a Retrovirus construct. In some embodiments the Retrovirus construct is derived from the Lentivirus. In some embodiments, transfecting/transducing the stem cells can be achieved through tagging the test gene and introducing the gene prepared according to methods known in the art such as those described in Miskinyte et al., Direct Conversion of Human Fibroblasts to Functional Excitatory Cortical Neurons Integrating Into Human Neural Networks, Stem Cell Research & Therapy (2017) 8:207. See e.g., the sections mentioned herein above.

In some embodiment the test gene is a gene from the human genome. In other embodiments the gene is not from the human genome. In some embodiments the test gene is a coding gene, while in others the test gene is a non-coding gene.

Still referring to FIG. 2. the method 200 further includes at process sequence 204 incubating the cell culturing system under conditions suitable to allow the one or more stem cells comprising the test gene to differentiate into a plurality of differentiated cells 204. In some embodiments the incubation of the cell culturing system lasts between 5-100 days, preferably 25-75 days, even more preferred is between 45 and 55 days. Other methods known in the art are in described in Miskinyte et al., Direct Conversion of Human Fibroblasts to Functional Excitatory Cortical Neurons Integrating into Human Neural Networks, Stem Cell Research & Therapy (2017) 8:207. In embodiments, during the culturing period the stem cells with the test gene can differentiate into subtype cells. In some embodiments the subtype cells can be excitatory, inhibitory neurons, astrocytes, oligodendrocyte or microglia, or other somatic cells. Once the cells are differentiated the cells can be harvested.

The method 200, further includes at process sequence 206 performing single cell RNA sequencing on the plurality of differentiated cells, wherein the single cell RNA sequencing of the plurality of differentiated cells is indicative of the test genes efficacy as a regulatory gene for cellular differentiation. Single cell RNA sequencing can be performed by methods known in the art and through methods described in Cuomo et al., Single-Cell RNA-sequencing of differentiating iPS cells reveals dynamic genetic effects on gene expression,” published Feb. 10, 2020. In embodiments analyzing the RNA sequencing data includes grouping all cells expressing the same test genes based on the barcodes. Then the cells expressing the test gene can be clustered using UMAP, t-SNE or similar. Each cluster of the cells can be classified into subtypes based on the genes highly expressed. Further, the analysis can be used to determine the effectiveness of the test gene in driving cellular differentiation.

In embodiments, the method of the present disclosure can test many genes and their random combinations for their impact on cell differentiation and development. Further, the RNA sequencing can be performed at different time points. The time variation may allow for quantifying the cell proportion and quantifying the speed of the cell differentiation. The time points can range from hours to days, or weeks.

In some embodiments the present disclosure relates to a method of identifying genes relating to cellular differentiation, the method including: contacting a plurality of stem cells with one or more tagged regulatory genes and a selection marker to form a first plurality of transfected/transduced stem cells; selecting the first plurality of transfected/transduced stem cells; culturing the plurality of transfected/transduced stem cells under conditions suitable to allow the plurality of transfected/transduced stem cells to differentiate into a plurality of differentiated cells expressing the one or more tagged regulatory genes; and performing a single cell RNA sequencing on the plurality of differentiated cells to identify genes relating to cellular differentiation. In some embodiments, the selection marker is an antibiotic selection marker. In some embodiments, isolating includes contacting the plurality of stem cells and the first plurality of transfected/transduced stem cells with an antibiotic in an amount sufficient to kill the plurality of stem cells or the untransfected/untransduced cells. In some embodiments, a pool of a plurality of retrovirus constructs delivers the one or more regulatory genes to the plurality of stem cells. In some embodiments, the plurality of retrovirus constructs are derived from Lentivirus. In some embodiments, the one or more tagged regulatory genes comprise a sequence including a 6-10 base pair barcode. In some embodiments, performing a single cell RNA sequencing on the plurality of differentiated cells to identify genes relating to cellular differentiation further comprises grouping the cells by gene expression profile. In some embodiments, performing a single cell RNA sequencing on the plurality of differentiated cells to identify genes relating to cellular differentiation further comprises clustering the cell cultures using UMAP or t-SNE; and classifying the cell cultures into a plurality of subtypes based on a primary regulatory gene. In some embodiments, determining a plurality of cell types formed. In some embodiments, determining the primary regulatory gene found in each of the plurality of cell types. In some embodiments, the one or more tagged regulatory genes include a gene found in the human genome. In some embodiments, the one or more genes are selected from the group consisting of coding and non-coding genes.

In some embodiments, the present disclosure relates to a method for identifying a regulatory gene relating to cellular differentiation, the method including: transfecting/transduced a plurality of stem cells within a cell culturing system with a test gene; incubating the cell culturing system under conditions suitable to allow the one or more stem cells including the test gene to differentiate into a plurality of differentiated cells; and performing single cell RNA sequencing on the plurality of differentiated cells, wherein the single cell RNA sequencing of the plurality of differentiated cells is indicative of the test gene efficacy as a regulatory gene for cellular differentiation. In some embodiments, the test gene is a gene from the human genome. In some embodiments, the methods include tagging the test gene; and delivering the test gene to the one or more stem cells via a Retrovirus.

In some embodiments, the present disclosure relates to a non-transitory computer readable medium such as memory having instructions stored thereon that, when executed, causes an apparatus to perform a method, including: contacting a plurality of stem cells with one or more tagged regulatory genes and a selection marker to form a first plurality of transfected/transduced stem cells; selecting the first plurality of transfected/transduced stem cells; culturing the plurality of transfected/transduced stem cells under conditions suitable to allow the plurality of transfected/transduced stem cells to differentiate into a plurality of differentiated cells expressing the one or more tagged regulatory genes; and performing a single cell RNA sequencing on the plurality of differentiated cells to identify genes relating to cellular differentiation.

The disclosure may be practices using RNA sequencing, and cell culturing systems wherein the parameters may be adjusted to achieve acceptable characteristics by those skilled in the art by utilizing the teachings disclosed herein.

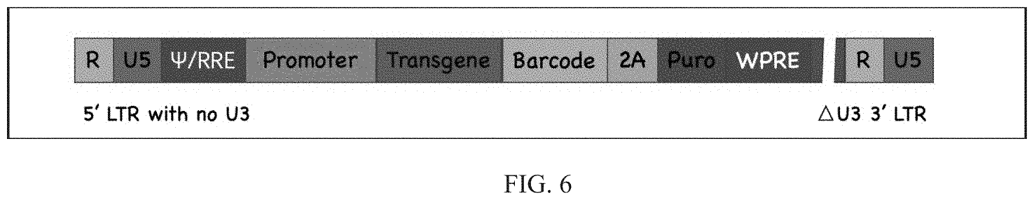

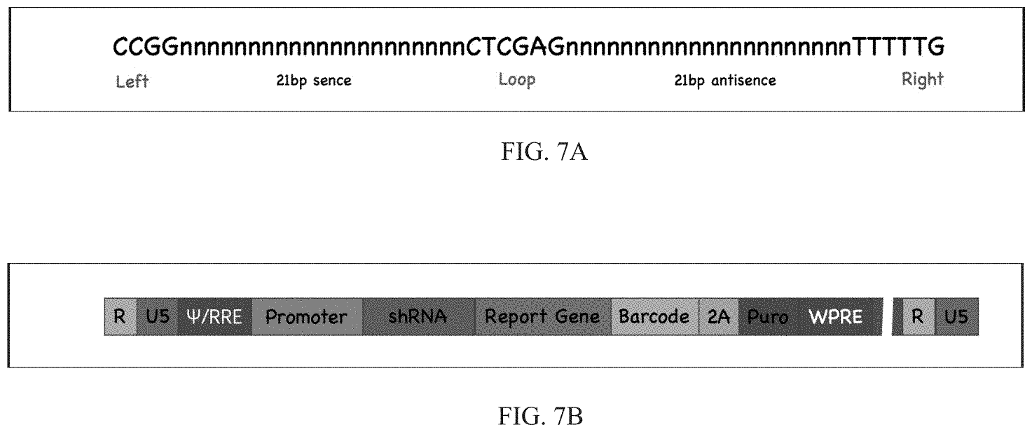

In embodiments, the present disclosure relates to one or more DNA constructs including a promoter upstream a predetermined shRNA, which is upstream a reporter-gene-of-interest, which is upstream a barcode sequence. In embodiments, the DNA constructs are transduced/transfected into a cell. In embodiments, the DNA construct is either transduced into a cell, or transfected into a cell, but not both. See e.g., FIGS. 6, 7A, and 7B depicting suitable DNA constructs for use in accordance with the present disclosure. In some cases, the barcode sequences are at least about 5 nucleotides in length. Also, the barcode sequences may be random polynucleotide sequences. In embodiments, barcodes can be attached to polynucleotides of the present disclosure by the methods described in U.S. Pat. No. 9,388,465 (herein entirely incorporated by reference).

In embodiments, sequence information is obtained in the form of sequence reads and obtained using a droplet based single-cell RNA-sequencing (scRNA-seq) microfluidics system that enables 3′ or 5′ messenger RNA (mRNA) digital counting of thousands of single second entities (e.g., single cells). In such sequencing, droplet-based platform enables barcoding of cells. See e.g., U.S. Pat. No. 10,347,365 (herein incorporated by reference) See also, U.S. Pat. No. 10,428,326. In embodiments, the microfluidic system includes software or non-transient computer readable media.

In embodiments, a GFP protein is provided as positive control in the process to monitor cell growth and differentiation. In embodiments, suitable reporter genes for use herein include (GFP, YFP, RFP, etc.) to monitor proportion of cells derived from cells with different transgenes.

In embodiments, the present disclosure includes an expression vector, including: a coding target gene for RNA sequencing, wherein the coding target gene comprises an untranslated leader sequence or an untranslated trailer sequence; and a 6 base-pair barcode attached to the untranslated leader sequence or the untranslated trailer sequence. In embodiments, the expression vector includes a coding target gene including only an untranslated trailer sequence, and the 6 base-pair barcode is attached to the untranslated trailer sequence. In embodiments, the coding target gene includes only an untranslated leader sequence, and the 6 base-pair barcode is attached to the untranslated leader sequence. In embodiments, the present disclosure includes a host cell including the expression vector of the present disclosure. In embodiments, an expression vector suitable for use herein includes the vector of FIG. 8, such as the vector described in Table I and the accompanying sequence listings.

### EXAMPLES

### Example I

In embodiments, the present disclosure includes one or more expression vectors including a promoter sequence, and a preselected nucleic acid construct including one or more genes-of-interest. An example of an expression vector suitable for use herein includes the expression vector of SEQ ID NO: 1. In embodiments, genes-of interest may include pre-selected candidate genes that have the potential to regulate cell differentiation from stem cells based on gene expression profiles, including but not limited to those reported in early fetal brains and iPSC-derived NPC and neurons. The present disclosure includes a Lentivirus vector, such as depicted in FIG. 8, which includes, inter alia, a target gene, e.g., such as ASCL1, wherein the vector is able to overexpress the target gene. In embodiments, the vector includes a reporter gene, such as DNA encoding EGFP fluorescence protein. In embodiments, the vector includes a barcode sequence, e.g., ACAGTG is as shown at the end of the target gene (ASCL1 in FIG. 8). In embodiments, the expression vector includes a promoter operably linked to a target gene. For example, as shown in FIG. 8, EF1A promoter is included to drive the expression of the target gene. In embodiments, the expression vector includes a selectable marker gene such as an Ampicillin resistant gene for screening of plasmid. Puromycin resistant gene (Puro) is used for screening transduced cells. In embodiments, the promoter sequence is operably linked to the nucleic acid construct. In embodiments, the promoter sequence is EF1A promoter.

In embodiments, the expression vector of the present disclosure is transduced or transformed into a host cell, such as one or more stem cells of the present disclosure.

Referring now to FIG. 8 and expression vector suitable for use herein is shown. In embodiments, the expression vector includes a gene-of-interest, or a gene to be investigated in accordance with the present disclosure. In embodiments, the vector includes the constituents as set forth in Table 1 below:

TABLE 1

| | | Size | | | |
|---|---|---|---|---|---|
| Name | Position | (bp) | Description | Function | SEQ ID NO |
| RSV promoter | 1-220 | 229 | Rous sarcoma virus | Strong | 3 |
| | | | enhancer/promoterNone | promoter; | |
| | | | | drives | |
| | | | | transcription of | |
| | | | | viral RNA in | |
| | | | | packaging | |
| | | | | cells. | |
| 5′ LTR-ΔU3 | 230-410 | 181 | Truncated HIV-1 5′ long | Allows | 4 |
| | | | terminal repeatNone | transcription of | |
| | | | | viral RNA and | |
| | | | | its packaging | |
| | | | | into virus. | |
| Ψ | 521-565 | 45 | HIV-1 packaging signal | Allows | 5 |
| | | | | packaging of | |
| | | | | viral RNA into | |
| | | | | virus. | |
| RRE | 1075- | 234 | HIV-1 Rev response | Rev protein | 6 |
| | 1308 | | element | binding site | |
| | | | | that allows | |
| | | | | Rev-dependent | |
| | | | | nuclear export | |
| | | | | of viral RNA | |
| | | | | during viral | |
| | | | | packaging. | |
| cPPT | 1803- | 118 | Central polypurine tract | Factates the | 7 |
| | 1920 | | | nuclear import | |
| | | | | of HIV-1 cDNA | |
| | | | | through a | |
| | | | | central DNA | |
| | | | | flap. | |
| EF1A | 1959- | 1179 | Human eukaryotic | Strong | 8 |
| | 3137 | | translation elongation | promoter | |
| | | | factor 1 α1 | | |
| | | | promoterNone | | |
| Kozak | 3162- | 6 | Kozak translation | Facilitates | 9 |
| | 3167 | | initiation sequence | translation | |
| | | | | initiation of | |
| | | | | ATG start | |
| | | | | codon | |
| | | | | downstream of | |
| | | | | the Kozak | |
| | | | | sequence. | |
| hASCL1 (or any | 3168- | 711 | Gene-of-interest | | 10 |
| gene of | 3878 | | | | |
| interest) | | | | | |
| barcode | 3879- | 6 | barcode | | 11 |
| | 3884 | | | | |
| WPRE | 3923- | 598 | Woodchuck hepatitis | Enhances virus | 12 |
| | 4520 | | virus posttranscriptional | stabiliy in | |
| | | | regulatory element | packaging | |
| | | | | cells, leading to | |
| | | | | higher titer of | |
| | | | | packaged | |
| | | | | virus; | |
| | | | | enhances | |
| | | | | higher | |
| | | | | expression of | |
| | | | | transgenes. | |
| CMV | 4542- | 588 | Human cytomegalovirus | Strong | 13 |
| PROMOTER | 5129 | | immediate early | promoter; may | |
| | | | enhancer/promoter | have variable | |
| | | | | strength in | |
| | | | | some cell | |
| | | | | types. | |
| EGFP:T2A:Puro | 5161- | 1380 | EGFP and Puro linked | Allows cells to | 14 |
| | 6540 | | by T2ANone | be visualized | |
| | | | | by green | |
| | | | | fluorescence | |
| | | | | and resistant to | |
| | | | | puromycin. | |
| 3′ LTR-ΔU3 | 6611- | 235 | Truncated HlV-1 3′ long | Allows | 15 |
| | 6845 | | terminal repeat | packaging of | |
| | | | | viral RNA into | |
| | | | | virus, self- | |
| | | | | inactivates the | |
| | | | | 5′ LTR by a | |
| | | | | copying | |
| | | | | mechanism | |
| | | | | during viral | |
| | | | | genome | |
| | | | | integration; | |
| | | | | contains | |
| | | | | poiyadenylation | |
| | | | | signal for | |
| | | | | transcription | |
| | | | | termination. | |
| SV40 early PA | 6918- | 135 | Simian virus 40 early | Allows | 16 |
| | 7052 | | polyadenyiation signal | transcription | |
| | | | | termination and | |
| | | | | polyadenylation | |
| | | | | of mRNA | |
| | | | | transcribed by | |
| | | | | Pol II RNA | |
| | | | | polymerase. | |
| Ampicillin | 8006- | 861 | Ampicillin resistance | Allows E. coli | 17 |
| | 8866 | | gene | to be resistant | |
| | | | | to ampicillin | |
| pUC ori | 9037- | 589 | pUC origin of | Facilitates | 18 |
| | 9625 | | replicationNone | plasmid | |
| | | | | replication in E. | |
| | | | | coli; regulates | |
| | | | | high-copy | |
| | | | | plasmid | |
| | | | | number (500- | |
| | | | | 700). | |

### Prophetic Example II

An Enhanced & Suppressed Expression triggered Cell Differentiation Sequencing (ESECD-seq) method is created which can perform high-throughput screening of genes that drive cell differentiation with reduced costs and much less labor. An innovative high throughput system is provided that takes advantage of snRNA-seq to identify cells transduced by viruses containing genes desired for overexpress or knockdown and tagged with barcodes. Simultaneously, the process of the present disclosure identifies the construct integrated into a cell, and the resulting neural cell type, by detecting and quantifying barcodes and marker genes. 20 or more candidate genes are screened in accordance with the present disclosure. In embodiments, between 10 and 1000, 10 and 1000 genes are screened in accordance with the present disclosure. In embodiments, between 10 and 50, 10-100, 10-1,000, 100-1000 candidate genes are screened in accordance with the present disclosure. In embodiments, between 10 and 100 candidate genes are screened in accordance with the present disclosure. In embodiments, between 10 and 100 candidate genes are screened in accordance with the present disclosure.

ESECD-seq of the present disclosure has several advantages compared with other procedures in the art. The inventors test the effects of suppressing candidate gene expression, which is complementary and represents a distinct type of regulation. In embodiments, the present disclosure uses snRNA-seq to capture internal expression markers of cell subtypes or all possible cell subtypes. A small number of genes is used to start and will provide excellent cell-type discrimination power. The ESECD-seq has a clear advantage of greater discrimination power because the methods of the present disclosure are not limited by antibody availability and/or unique surface-expressed proteins.

In embodiments, major research gaps are filled such as: 1) unknown biological functions of many genetic findings of SCZ; 2) unknown genes that can drive neural cell differentiation from stem cells. Conceptually, the inventors observe that certain insults early in pregnancy are associated with risk of developing schizophrenia (SCZ). Altered expression of critical genes in the first few days or months of brain development may have consequences such as SCZ later in life. The identity of those critical genes is unknown. In embodiments, the present disclosure uses an hESCs to model the effects of expression changes. In embodiments, the present disclosure uses an iPSC to model the effects of expression changes. Cell differentiation of stem cells is accompanied by expression changes, driven by changes of key regulators.

### Approaches

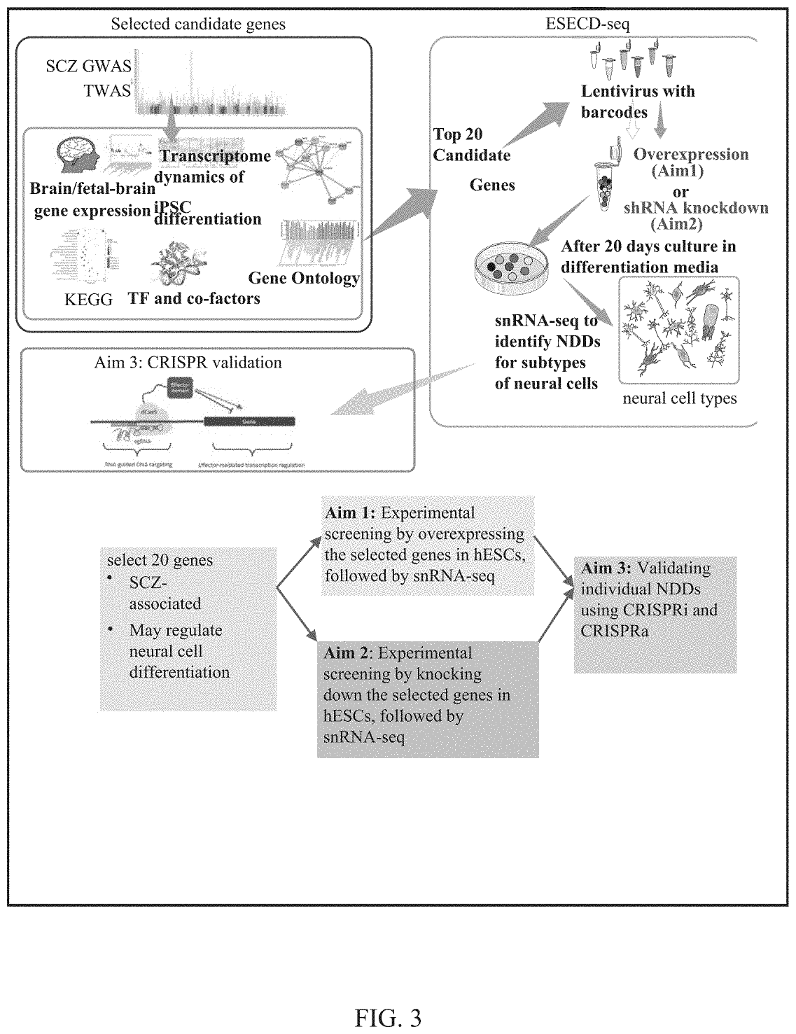

The overall process flow is shown in FIG. 3. Twenty candidate genes that are all associated with schizophrenia (SCZ) are selected for testing in accordance with the present disclosure. Many of the genes selected for this test either regulate cell differentiation, or not, when they are over-expressed. Several untested genes are also included. This test will serve to validate the ESECD-seq system. Aims 1 and 2 use complementary approaches to experimentally test these candidates for their ability to drive hESC to differentiate into neural cell types. Aim 3 uses CRISPRa and CRISPRi to individually validate the discovered neural differentiation drivers from Aims 1 and 2.

### Gene Selection

In embodiments, the present disclosure increases the rate at which genes can be screened for their potential to influence cell differentiation. Initial efforts are conservative, screening 20 genes, some of which have preliminary evidence suggesting their involvement in cell differentiation. More genes whose involvement in cell differentiation is completely unknown will be tested.

Genome-wide association studies (GWAS) identified 179 SNPs significantly associated with schizophrenia (SCZ), and these SNPs implied 731 genes. See e.g., Pardiñas A F, et al., Common schizophrenia alleles are enriched in mutationintolerant genes and in regions under strong background selection. Nature Genetics. 2018; 50(3):381-9. doi: 10.1038/s41588-018-0059-2; PMCID: PMC5918692. Besides a few genes that are related to neurotransmitters, ion channels, and immunity, most of the genes have no apparent functions that are related to SCZ etiology. In addition to genes identified in GWAS, there are also many genes associated with SCZ by de novo mutations, (See e.g., Howrigan D P, et al., Schizophrenia risk conferred by protein-coding <em>de novo</em> mutations. bioRxiv. 2018:495036. doi: 10.1101/495036; Kranz T M, et al. De novo mutations from sporadic schizophrenia cases highlight important signaling genes in an independent sample. Schizophr Res. 2015; 166(1-3):119-24. Epub 2015/06/21. doi: 10.1016/j.schres.2015.05.042. PubMed PMID: 26091878; PMCID: PMC4512856; and Li J, et al., Genes with de novo mutations are shared by four neuropsychiatric disorders discovered from NPdenovo database. Mol Psychiatry. 2016; 21(2):290-7. Epub 2015/04/08. doi: 10.1038/mp.2015.40. PubMed PMID: 25849321; PMCID: PMC4837654) copy number variants, and transcriptome-wide associations (TWASs). (See e.g., Gusev A, et al. Transcriptome-wide association study of schizophrenia and chromatin activity yields mechanistic disease insights. Nat Genet. 2018; 50(4):538-48. Epub 2018/04/11. doi: 10.1038/s41588-018-0092-1. PubMed PMID: 29632383; PMCID: PMC5942893; Hall L S, et al. A transcriptome-wide association study implicates specific pre- and post-synaptic abnormalities in schizophrenia. Hum Mol Genet. 2020; 29(1):159-67. Epub 2019/11/07. doi: 10.1093/hmg/ddz253. PubMed PMID: 31691811). The inventor opts to focus on GWAS signals as they are the most credible to date. Out of the 20 candidate genes the inventor selected for this trial, Church's group tested 13 of them, and found 6 to be able to drive differentiation to neurons by overexpression of a single gene (Table 2).

TABLE 2
Table 1. Candidate Genes for ESECD-seq

| | | | Positive in |
|---|---|---|---|
| Symbol | Module | TF_family | Church's study |
| ASCL1* | 1 | bHLH | Yes |
| PBRM1 | 1 | HMG | |
| RERE | 1 | zf-GATA | |
| CPEB1 | 1 | Others | |
| ZSCAN 2 | 1 | zf-C2H2 | |
| ZNF536 | 1 | zf-C2H2 | Yes |
| BCL11B | 1 | zf-C2H2 | |
| PBX4 | 1 | Homeobox | Yes |
| ZNF491 | 1 | zf-C2H2 | Yes |
| SATB2 | 2 | CUT | |
| ARNT | 2 | bHLH | |
| GABPB2 | 2 | Others | |
| SREBF1 | 2 | bHLH | |
| SETDB1 | 2 | MBD | |
| NFATC3 | 2 | RHD | |
| ZNF440 | 2 | zf-C2H2 | Yes |
| TCF4 | 3 | bHLH | |
| STAT6 | 3 | STAT | Yes |
| TBX6 | 3 | T-box | |
| N R1H 3 | 3 | THR-like | Yes |

All these genes are SCZGWAS signals
*positive control;
Module: coexpression modules by Burke et al. refer to FIG. 2.A
**Church+3 s study refers to the study disclosed in Ng, A.H.M., Khoshakhlagh, P., Rojo Arias, J. E. et al. A comprehensive library of human transcription factors for cell fate engineering. Nat Biotechnol 39, 510-519 (2021). https://doi.org/10.1038/s41587-020-0742-6 (herein entirely incorporated by reference).

In embodiments, the other 7 genes do not show activity driving cell differentiation. Selection of genes known to be, and not be, involved in differentiation provides the opportunity to use Church's results as a benchmark for our ESECD-seq. It is expected that the genes shown to be neural differential drivers (NDDs) in Church's study referenced above should also be determined to be NDDs by ESECD-seq. Genes called negative in Church's study still have chance to be detected as NDDs in this study, as ESECD-seq is able to assess more cell types for differentiation driven by both overexpression and suppression of the target genes.

Table 1 shows a list of 20 candidates identified based on the analyses of the 731 genes from GWAS associated regions. Several positive controls are included, including Ascl1 which is well-known for its ability to differentiate hESC. 6 NDDs are included discovered by Church's group in overexpression screening. Seven genes shown by Church to not be associated with differentiation were included, as well as 5 genes that were not tested by Church. A negative control is also used (details in D.2).

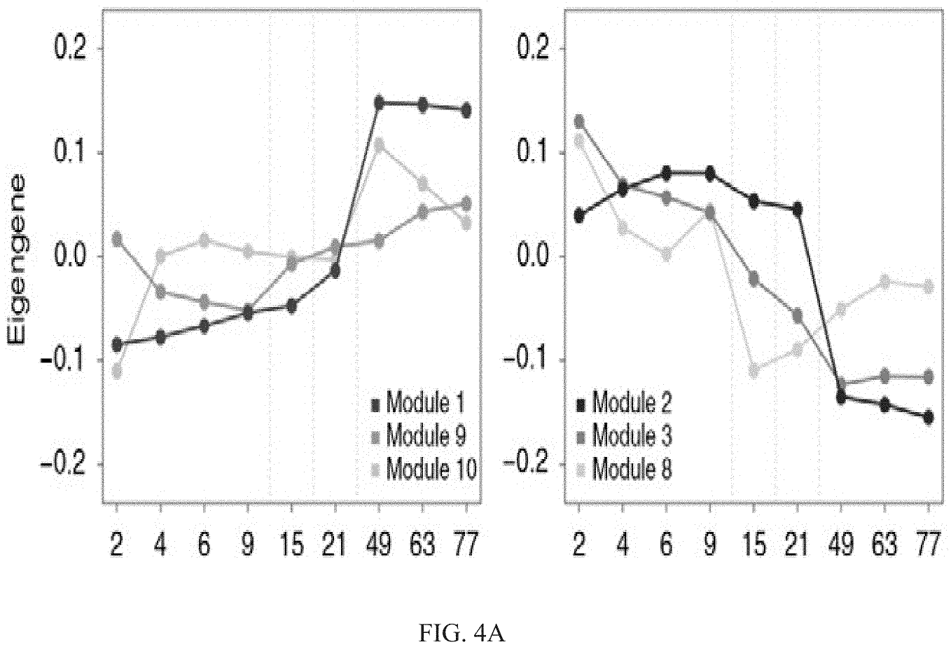

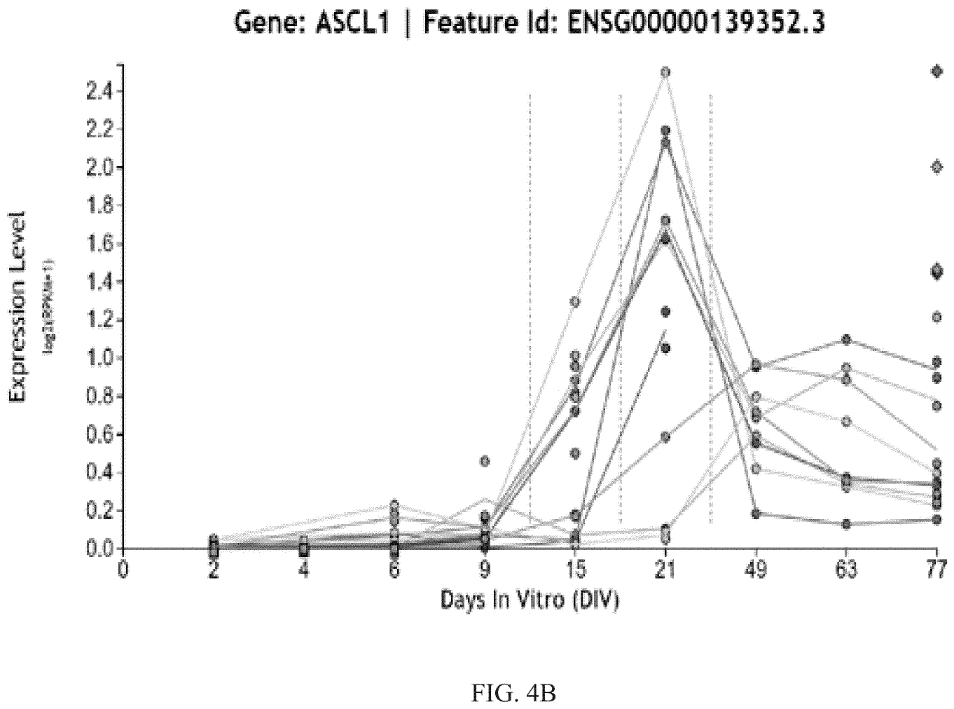

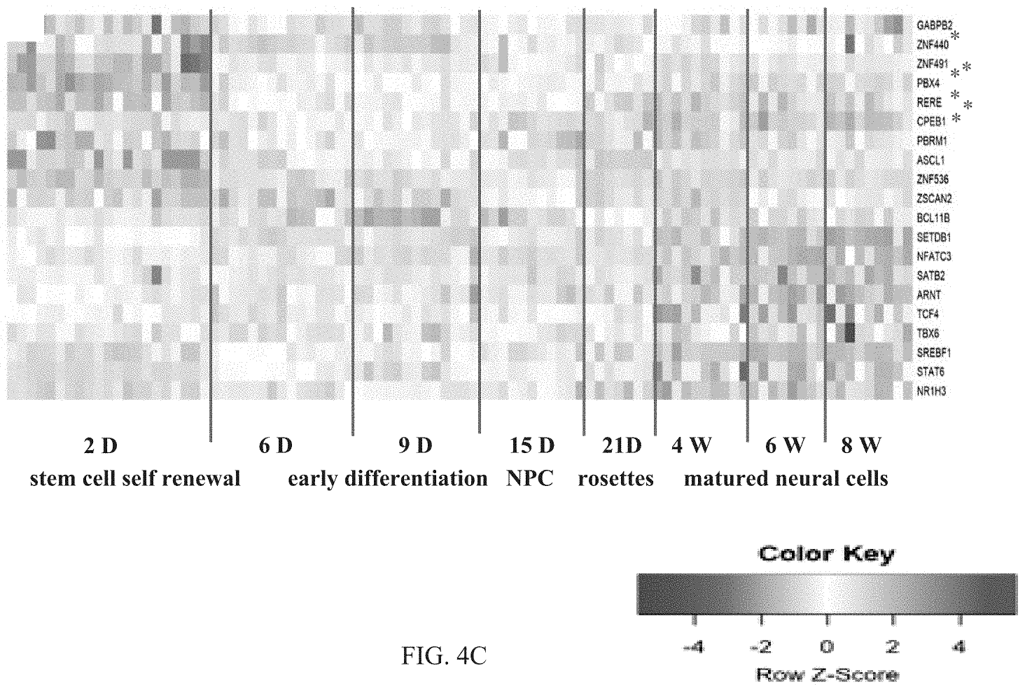

In addition to regulators being more likely to be TFs or co-factors, the inventors have discovered that the genes with regulation potential have specific time-dependent expression patterns (FIG. 4B for Ascl1 as an example). Based on these signatures, a list of candidate genes was compiled with additional filters on SCZ-associated genes according to: 1) Gene Ontology and KEGG pathway data for known TFs and co-factors; 2) Transcriptome dynamics data of iPSC differentiation into neurons (See e.g., Burke E E, Dissecting transcriptomic signatures of neuronal differentiation and maturation using iPSCs. Nat Commun. 2020; 11(1):462. Epub 2020/01/25. doi: 10.1038/s41467-019-14266-z. PubMed PMID: 31974374; PMCID: PMC6978526), coexpressed with known NDDs like Ascl1. as shown in FIG. 4A. FIG. 4C shows the expression profiles of the 20 selected genes in the transcriptome changes when iPSCs differentiate to neurons. One group of genes increase expression over time, another decreases, suggesting possible effects of the knockdown and overexpression in our ESECD-seq.

D.2. Aim 1. ESECD-seq to screen for over-expressed genes that are capable of driving differentiation of hESCs to any subtype of neural cells.

A pool of barcoded lentivirus constructs is used to transduce the 20 selected genes into six hESC lines originating from three male and three female donors. The detailed procedure of Aim1 is shown in FIG. 3. After transduction, culture and antibiotic screening, snRNA-seq will be used to identify neural cell types using established marker genes. Through data analysis, the transduced genes will be directly related to the differentiated cells. This Aim will identify overexpressed genes that can drive hESC differentiation.

D.2.1. Creating Pools of Transgenic hESCs for the 20 Candidate Genes.

D.2.1.a hESCs and Quality Control:

This study uses six hESCs from donors of 3 healthy males and 3 healthy age-matched females from NIH Human Embryonic Stem Cell Registry (Male: WA01 (H1), WA14 (H14), WA17; Female: WA07 (H7), WA09 (H9), WA21).

Cells are subjected to rigorous quality control procedures based on established protocols (See e.g., D'Antonio M, et al., High-Throughput and Cost-Effective Characterization of Induced Pluripotent Stem Cells. Stem Cell Reports. 2017; 8(4):1101-11. doi: 10.1016/j.stemcr.2017.03.011; PMCID: PMC5390243, and Sullivan S, et al. Quality control guidelines for clinical-grade human induced pluripotent stem cell lines. Regenerative Medicine. 2018; 13(7):859-66. doi: 10.2217/rme-2018-0095) to ensure lines are stable and pluripotent. The hESCs are thoroughly characterized to be sure they are free of mycoplasma, homogeneous, pluripotent, and are genetically stable periodically during cell maintenance and just prior using them in experiments.

1) Contamination test. Mycoplasma testing are completed using an Applied Biosystems Real-time PCR mycoplasma testing kit.

2) Validating the pluripotency of hESCs is vital to the success of the experiment because the inventors are interested in determining if genes being tested can cause differentiation to other cell types. The TaqMan hPSC Scorecard (ThermoFisher) will be used in this experiment because it is simple, fast, and reliable. Homogeneity will be tested by immunocytochemistry every third passage during cell maintenance.

3) Genetic stability of hESCs will be assessed using a StemCell Technologies qPCR-based hPSC genetic analysis kit.

D.2.1.b hESC Maintenance:

hESCs are grown using commercial media by StemCell Technologies. Cells will be started and grown on Matrigel-coated plates through the entire duration of the experiment in mTeSR Plus feeder-free medium. Cells are split using ReLeSR, which lifts only undifferentiated cells.

D.2.1.c Lentivirus Construction and Validation:

Third generation lentivirus constructs are designed to constitutively over-express genes, as shown in FIG. 6. Referring to FIG. 6, an overexpression lentivirus construction for the transfer plasmid is shown. A typical LTR (long terminal repeat) includes three virus elements, U3-R-U5. In this vector, the 5′ LTR does not contain U3. The 3′ LTR has U3 mutated. RRE is a Rev response element, with a strong promoter like CMV. A 6 bp barcode is used at the 3′ UTR of transgene. 2A is self-cleaving peptides and Puromycin is an antibiotic protein, Posttranscriptional Regulatory Element (WPRE) enhances the expression of transgenes by increasing nuclear export.

The 20 genes selected from Aim 1 are introduced into constructs. The candidate genes will be tagged, each with a unique 6 bp barcode at its 3′ UTR. The barcode will be transcribed to serve as identifiers of the transgenes in the transcriptome of the transduced cells. Lentiviruses will be purchased from Viraquest or Welgen.

Positive controls use Ascl1 as transgenes since they are known to drive stem cell differentiation. (See e.g Pang Z P et al., Induction of human neuronal cells by defined transcription factors. Nature. 2011; 476(7359):220-3. Epub 2011/05/28. doi: 10.1038/nature10202. PubMed PMID: 21617644; PMCID: PMC3159048; Yang N et al., Generation of pure GABAergic neurons by transcription factor programming. Nat Methods. 2017; 14(6):621-8. Epub 2017/05/16. doi: 10.1038/nmeth.4291. PubMed PMID: 28504679; PMCID: PMC5567689; and Zhang Y, Rapid single-step induction of functional neurons from human pluripotent stem cells. Neuron. 2013; 78(5):785-98. Epub 2013/06/15. doi: 10.1016/j.neuron.2013.05.029. PubMed PMID: 23764284; PMCID: PMC3751803.). The positive control is used to validate that the cells are capable of differentiating to neuronal cells. A negative control will use an empty vector for baseline measure of cell differentiation.

Pilot experiments optimize the multiplicity of infection (MOI) using a lentivirus vector with GFP. hESCs is lifted and single cell suspensions will be counted, virus is added, and cells are plated in 3.5 cm dishes at a density of 300,000 to 400,000 cells per plate. Four days after transduction, cell counts are obtained. The MOI yielding the largest number of surviving cells is selected for further use.

D.2.1.d Transduction.

To transduce the hESCs, the viruses of all 20 transgenes, along with the negative control, are pooled and applied to cells using a MOI for each virus that is 1/21 of the optimum MOI. The goal is to provide each virus with an equal probability of transducing cells.

The virus pool is added on Day 0 to cells growing in mTeSR Plus media in 6-well plates at a density of 300,000 to 400,000 cells per well. Media is changed on day 2 to mTeSR Plus with puromycin, which will be replaced daily for four days so that only the transduced cells that express at least one transgene can survive. The hESCs with the correct overexpressed genes will differentiate into cell subtypes. Culturing the transduced cells is performed for a duration of two weeks with media changed daily. Cells are harvested for snRNA-seq on Day 20. This procedure will allow the growth of all major neural cell types, neuronal, and glial cells.

D.2.2. SnRNA-seq.

Cells will be harvested according to the 10× Genomics® protocol on “Single Cell Suspensions for Cultured Cell Lines for Single Cell RNA Sequencing.” Herein incorporated by reference. See e.g., https://support.10×genomics.com/single-cell-gene-expression/sample-prep/doc/demonstrated-protocol-single-cell-suspensions-from-cultured-cell-lines-for-single-cell-rna-sequencing. In particular, the general materials, preparation-buffers & media, single Cell Suspensions from Cultured Cell Lines, Cell Harvesting—Suspension Cell Lines, and Cell Harvesting—Adherence Cell lines descriptions are herein incorporated by reference. Trypsin-EDTA are used to lift cells, followed by incubation, halting the trypsin solution, and centrifugation. Cells are resuspended using culture medium, strained, and counted. After counting, cells undergo a series of washing steps and be counted to determine a final concentration. Nuclei isolation will follow this, according to the 10× Genomics® protocol on Isolation of Nuclei for Single Cell RNA Sequencing. See e.g., https://support.10×genomics.com/single-cell-gene-expression/sample-prep/doc/demonstrated-protocol-isolation-of-nuclei-for-single-cell-ma-sequencing. This protocol is herein incorporated by reference, including the best practices and general protocols for cell lysis, washing, debris removal, counting, and concentrating nuclei from both single cell suspensions and neural tissue in preparation for use in 10× Genomics® Single Cell Protocols. Cells are centrifuged and lysed with a lysis buffer. After cells are lysed, nuclei are centrifuged, washed, stained, and counted. Once a target concentration is obtained, nuclei are loaded onto a Chromium Next GEM Chip G, according to the Chromium Next GEM Single Cell 3′ Reagent Kits v3.1 User Guide. The Chromium machine will be used to prepare sequencing libraries. Sequencing is run on NextSeq 500 sequencer, which generates 500 million pair-end reads of 91-base, including 16-base barcode and 12-base UMI reads.

### D.2.3. Data Analyses.

D.2.3.a Cell Type Identification.

Raw sequencing data is processed using the 10× Genomics Cell Ranger v4.0 pipeline. Samples are demultiplexed and data is converted to Fastq format. The template switch oligo (TSO) sequence from the 5′ end and the poly-A sequence from the 3′ end will be removed from cDNA reads. Trimmed cDNA reads are aligned to human Gencode v32 reference genome using Orbit aligner. UMI counts for each gene with annotation is generated for each cell.

The processed count data is imported to Seurat v3.0. (See e.g, Stuart T, et al., Comprehensive Integration of Single-Cell Data. Cell. 2019; 177(7):1888-902 e21. Epub 2019/06/11. doi: 10.1016/j.cell.2019.05.031. PubMed PMID: 31178118; PMCID: PMC6687398). Multiple quality control plots is generated. Gene expression data is kept for cells with 300 to 3,000 genes expressed and genes expressed in at least 1% cells. Then cells are grouped according to the barcodes in constructs and analyzed separately. The data for each group expressing the same transgene(s) is normalized and transformed by SCtransformation. (See e.g., Hafemeister C, Satija R., Normalization and variance stabilization of single-cell RNA-seq data using regularized negative binomial regression. Genome Biol. 2019; 20(1):296. Epub 2019/12/25. doi: 10.1186/s13059-019-1874-1. PubMed PMID: 31870423; PMCID: PMC6927181).

The top 3,000 most variable genes out of all genes detected are selected for cell clustering visualization using UMAP. Each cell cluster is classified into subtypes by their transcriptome signature according to the marker genes of all major cell subtypes (Table 3).

TABLE 3
Table 2. Marker genes for the major neural cell types.

| Cell Type | Marker Genes |
|---|---|
| Neurons | GAD1, RTN1, GPRIN1, DCX, PRKAR1B, |
| | RBFOX3, SLC32A1, Kctd12 |
| Microglia | ITGAM, PTPRC, AIF1, TLR2, TLR7, CTSC |
| Astrocytes | ALDOC, CLU, SLC4A4, |
| | ALDH1L1, GJA1 |
| Oligodendrocytes | PLP1, ENPP6, LGI3, MBP, SLC44A1, CNP |

Correlations of the expression profile of each cell group with published snRNA-seq data of major neural cell subtypes is also tested to further confirm the identity of cell clusters. (See e.g, Mathys H, et al., Single-cell transcriptomic analysis of Alzheimer's disease. Nature. 2019; 570(7761):332-7. Epub 2019/05/03. doi: 10.1038/s41586-019-1195-2. PubMed PMID: 31042697; PMCID: PMC6865822; and Velmeshev D, et al., Single-cell genomics identifies cell type-specific molecular changes in autism. Science. 2019; 364(6441):685-9. Epub 2019/05/18. doi: 10.1126/science.aav8130. PubMed PMID: 31097668). snRNA-seq of fetal brain captures dozens of subtypes of neural cells that can serve as a reference panel.

D.2.3.b Barcodes Connect Cell Types to the Transgenes.

When processing snRNA-seq data, cells are grouped by the barcodes detected in transcripts. Therefore, the cell types of these differentiated cell groups are induced by the transgenes they carry and tagged by the barcodes.

The cells carrying the negative control (empty vector with only a barcode) will serve as the reference of baseline activity of differentiation. It is expected that hESC will have slow natural differentiation during the culture process and produce a very small number of differentiated cells without strong regulating genes. Therefore, cell groups with the amounts of differentiated cells similar to the negative control are discarded.

D.2.4. Confirmation of snRNA-Seq Screening Results.

The identified genes from the screening are validated. Differentiated cells are fixed in 4% paraformaldehyde, treated with antibodies unique to the particular neural cell type as found by the snRNA-seq, and verified by fluorescent signals by microscopy. NeuN, TUJ1, and SYNAPSIN is used for neurons, GFAP and s100β for Astrocytes, PDGF and NG2 for OPC, Olig2 and MBP for oligodendrocytes, Iba1 and TMEM119 for microglia.

D.2.5. Statistical power. The statistical power question here is about the possibility to detect positives in each cell line. It is a matter whether one can detect it or not. No covariate, including sex variable, or multiple testing problem involves. It is expected to sequence 500 M reads for each cell line and detect an average of 2,000 genes per cell for approximately 4,000 cells. Expression levels of marker genes of neural cells in existing snRNA-seq data is analyzed (See e.g., Velmeshev D, Schirmer L, Jung D, Haeussler M, Perez Y, Mayer S, Bhaduri A, Goyal N, Rowitch D H, Kriegstein A R. Single-cell genomics identifies cell type-specific molecular changes in autism. Science. 2019; 364(6441):685-9. Epub 2019/05/18. doi: 10.1126/science.aav8130. PubMed PMID: 31097668) and it is found that the top 1,000 detected genes can provide high confidence (p<1e-3) calls of major neural cell subtypes including excitatory and inhibitory neurons, oligodendrocytes and astrocytes. When the number of detected genes increased to 2,000, microglial cells could be resolved with high confidence. Based on this estimate, ESECD-seq of the present disclosure has 95% power to detect 5% out of all the cultured cells as differentiated cells driven by one of the twenty candidate genes, assuming all genes have an equal chance of transduction and a minimum 80 of the 2,000 cells carry the marker genes of corresponding cell types. Each cell line is evaluated separately. Each sex has three replicate lines. A total of six lines for cross-validation.

D.2.6. Expected Outcome.

It is expected that most cells will carry one of the transgenes; a small number of cells will take a random combination of two genes; and, even fewer will hold a random combination of three genes or more. Overexpression of six of the transgenes are expected to result in differentiation of hESCs to neuronal cells, while the rest of them may or may not differentiate hESCs into other cell types. Some combinations of genes differentiate hESCs into one specific cell subtype, and others to multiple cell subtypes. This result would imply that these genes may also act in the earliest developing brain.

Aim 2. To determine if suppression of selected genes promotes differentiation of hESCs to subtypes of neural cells.

A complementary approach to Aim 1 is provided, using shRNA knockdown to screen the same set of 20 candidate genes. This Aim identifies genes that, when down-regulated, can drive hESC differentiation. The experimental procedure is very similar to Aim 1 except for the lentivirus construct design. shRNA is introduced that suppress the target gene, along with a GFP and shRNA-specific barcode (FIGS. 7A and 7B). Referring to FIGS. 7A and 7B, a lentivirus construct for shRNA knockdown screening is shown. FIG. 7A depicts shRNA design of the present disclosure, wherein CCGG is AgeI site for ligation, TTTTTG is ensure that after shRNA transcription, the end sequence is UUUU, and CTCGAG is a loop sequence. The chain of a refers to the sequence specific to the target. Referring to FIG. 7B, the figure depicts components of the lentivirus transfer plasmid, with similar component as the overexpression vector (FIG. 4) except that w shRNA is suitable to target the candidate gene. GFP is used as the report gene with a barcode, which is the shRNA-specific tag.

D.3.1. shRNA Constructs.

A GFP and shRNA-specific barcode are linked at the 3′ end of GFP sequence. Lentivirus delivery of the shRNA enables stable expression and permanent knockdown of target genes. ShRNA is processed in the cell by Dicer and RISC/AGO2 complex. (See e.g., Paroo Z, Liu Q, Wang X. Biochemical mechanisms of the RNA-induced silencing complex. Cell Res. 2007; 17(3):187-94. Epub 2007/02/21. doi: 10.1038/sj.cr.7310148. PubMed PMID: 17310219).

As illustrated in FIG. 7A, a palindromic loop (CTCGAG) is used to form the stem loop hairpin structure of shRNA, and CCGG is the AgeI site for ligation. A GFP is fused with an shRNA-specific barcode as an indicator of shRNA transduction into cells (FIG. 7B).

No known gene with reduced expression drives stem cell differentiation into neural cell to date. Therefore, a positive control specific for this Aim is not present. The negative control incudes a scrambled sequence.

Referring to FIGS. 7A and 7B, a suitable Lentivirus construct for shRNA knockdown screening is depicted. FIG. 7A depicts shRNA design, CCGG is AgeI site for ligation, TTTTTG is ensure that after shRNA transcription, the end sequence is UUUU, and CTCGAG is a loop sequence. The chain of a refers to the sequence specific to the target. FIG. 7B depicts components of the lentivirus transfer plasmid, with similar component as the overexpression vector (FIGS. 4A-4C) except that we have here shRNA to target the candidate gene. GFP is used as the report gene with a barcode, which is the shRNA-specific tag.

Referring now to FIG. 4, FIG. 4 depicts the expression dynamics of candidate genes in iPSC-derived cells. More specifically, FIG. 4A depicts expression modules from Burke, et al. 2020, FIG. 4.b indicating the module 1, 2, and 3 where all of our candidate genes belong to. FIG. 4B depicts positive control gene Ascl1 expression over time. FIG. 4C depicts a heatmap of candidate gene expression in Burke et al. 2020. ** refers to a gene detected as NDDs by Church's study.

D.3.2. shRNA Transduction and Cell Culture.

The transduction and cell culture will be identical to Aim 1 described above.

D.3.3. snRNA-Seq and Data Analysis.

The procedure used in this Aim is similar to Aim 1, except that the barcode will be linked to GFP instead of the target transgene. The GFP used here is for producing a barcoded transcript that is long enough to be detected in snRNA-seq. shRNA per se is too short for RNA-seq to catch. Cell type identification and the barcode-facilitated gene-cell-type connection is done in the same way as Aim 1.

D.3.4. Confirmation of snRNA-Seq Results.

The identified genes from the screening is individually validated by a single lentivirus shRNA assay, followed by fluorescent antibody staining with microscopy. Electrophysiology recording will be used to verify the function of differentiated neurons as well.

In the validation of knockdown, the concern of the off-target effect is addressed by using a second independent shRNA design.

D.3.5. Expected Outcome.

The expected outcome is that downregulation of one or more of the candidate genes causes hESCs to differentiate to some type of neural cell. This result implies that the gene or genes could be involved in cell differentiation in the early developing brain.

D.4. Sex and individual variation analyses for Aims 1 and 2

D.4.1. Sex Effects.

Since we have hESC from three males and three females, sex-related differences for the genes' ability to drive differentiation is analyzed.

D.4.2. hESC Donor Differences.

For both Aims 1 and 2, individual differences among donors is assessed. Heterogeneity in cellular phenotypes may arise from a variety of sources such as genetic variation among donors, variation in clones within donors, and culture protocols. (See e.g., Schwartzentruber J, Foskolou S, Kilpinen H, Rodrigues J, Alasoo K, Knights A, Patel M, Goncalves A, Ferreira R, al. e. Molecular and functional variation in iPSC-derived sensory neurons. Nature Genetics. 2018; 50(1):54-61). The range in percentage of variation in differentiation capacity among hESCs due to different donors has been reported to be 5-46%. (See e.g., Kilpinen H, Goncalves A, Leha A, Afzal V, Alasoo K, Ashford S, Bala S, Bensaddek D, Casale F P, al. e. Common genetic variation drives molecular heterogeneity in human iPSCs. Nature. 2017; 546(7658):370-5). If large differences in differentiation capacity are detected among hESC lines, we will investigate the causes closely by comparing expression levels of constructs and other genes associated with differentiation.

D.5. Aim 3. Validation of NDDs discovered from Aims 1 and 2 using single-gene CRISPRi and CRISPRa assay on hESCs followed by immuno-staining with cell-type-specific marker genes. CRISPRi and CRISPRa will be used to suppress or activate target gene expression. Both CRISPRa and CRISPRi use the enzymatically deficient Cas9 (dCas9), which is fused with expression activator or repressor. (See e.g., Gilbert L A, Horlbeck M A, Adamson B, Villalta J E, Chen Y, Whitehead E H, Guimaraes C, Panning B, Ploegh H L, Bassik M C, Qi L S, Kampmann M, Weissman J S. Genome-Scale CRISPR-Mediated Control of Gene Repression and Activation. Cell. 2014; 159(3):647-61. Epub 2014/10/14. doi: 10.1016/j.cell.2014.09.029. PubMed PMID: 25307932; PMCID: PMC4253859). With guide RNA (gRNA), the dCas9 complex target gene promoter to regulate gene expression. Antibody-based cell staining will be used to characterize and quantify the differentiated subtypes of cells. Therefore, we have an independent validation of the regulatory effect of the discovered NDD.

CRISPRa is used to validate NDDs identified from Aim 1. Instead of introducing an additional exogenous gene, CRISPRa enhances endogenous gene expression. The OriGene Cas9 is used for synergistic activation mediators complex (Cas9-SAM) pCas-Guide-CRISPRa vector, with the gRNA targeting the gene to be validated. Lentiviral delivery of the construct and subsequent antibiotic selection is used.

CRISPRi will be used to validate all the NDDs discovered from Aim 2. The OriGene pCas-Guide-CRISPRi vector is used, which has dCas9 fused with KRAB and MeCP2 repression domains to repress target gene repression, guided by the gRNA. The lentiviral transduction and antibiotic selection procedures are identical to the CRISPRa.

The differentiated cells are characterized by selected antibody according to the cell types identified in Aims 1 and 2, and subsequently counted microscopically. QCPR is used to assess target gene expression. Cell differentiation measured by the cell count of target cell type is tested for correlation with gene expression level.

Both CRISPRa and CRISPRi are performed in three replicates.

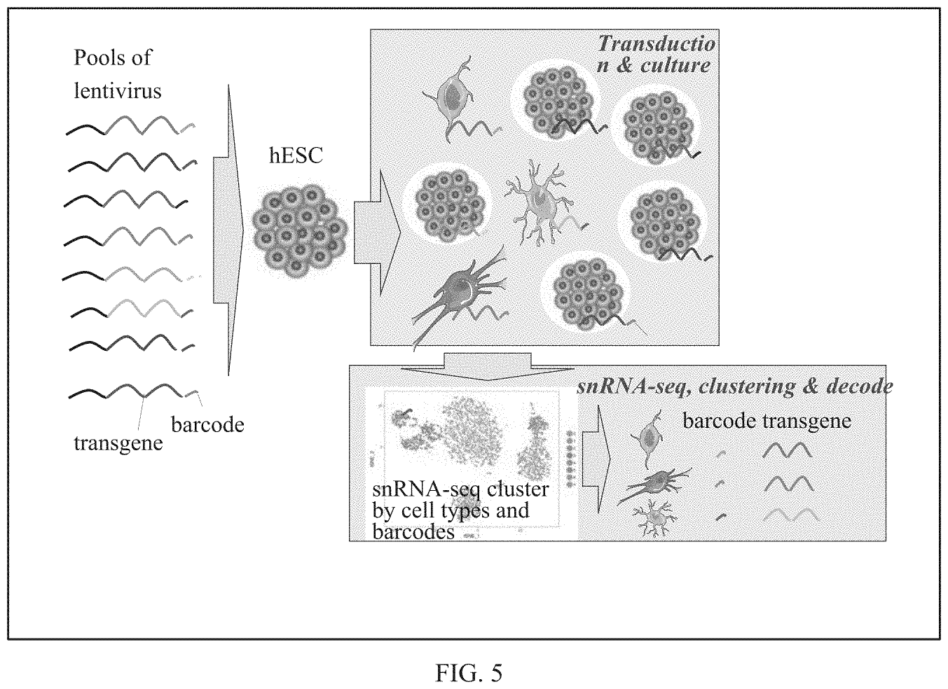

Referring to the Figures, FIG. 5 depicts coding and decoding of genes that can induce hESC differentiation. FIG. 6 depicts overexpression lentivirus construction for the transfer plasmid. A typical LTR (long terminal repeat) includes three virus elements, U3-R-U5. In this vector, the 5′ LTR does not contain U3. The 3′ LTR has U3 mutated. RRE is a Rev response element, with a strong promoter like CMV. A 6 bp barcode at the 3′ UTR of transgene is suitable for use herein. Still referring to FIG. 6, 2A is self-cleaving peptides and Puromycin is an antibiotic protein, Posttranscriptional Regulatory Element (WPRE) enhances the expression of transgenes by increasing nuclear export.

FIG. 8 depicts an expression vector suitable for use in accordance with the present disclosure. In embodiments, the expression vector is suitable of transfecting or transducing into a host cell, such as a preselected stem cell.

The entire disclosure of all applications, patents, and publications cited herein are herein incorporated by reference in their entirety. While the foregoing is directed to embodiments of the present disclosure, other and further embodiments of the disclosure may be devised without departing from the basic scope thereof.

What is claimed:

1. A method of identifying genes relating to cellular differentiation, the method comprising:
contacting a plurality of stem cells with one or more tagged regulatory genes and a selection marker to form a first plurality of transfected/transduced stem cells;
selecting the first plurality of transfected/transduced stem cells;
culturing the first plurality of transfected/transduced stem cells under conditions suitable to allow the first plurality of transfected/transduced stem cells to differentiate into a plurality of differentiated cells expressing the one or more tagged regulatory genes; and
performing a single cell RNA sequencing on the plurality of differentiated cells to identify genes relating to cellular differentiation.

2. The method of claim 1, wherein the selection marker is an antibiotic selection marker.

3. The method of claim 1, wherein isolating comprises contacting the plurality of stem cells and the first plurality of transfected/transduced stem cells with an antibiotic in an amount sufficient to kill the plurality of stem cells.

4. The method of claim 1, wherein a pool of a plurality of Retrovirus constructs delivers the one or more regulatory genes to the plurality of stem cells.

5. The method of claim 4 wherein the plurality of Retrovirus constructs are derived from Lentivirus.

6. The method of claim 1, wherein the one or more tagged regulatory genes comprise a sequence comprising a 6-10 base pair barcode.

7. The method of claim 1, wherein performing a single cell RNA sequencing on the plurality of differentiated cells to identify genes relating to cellular differentiation further comprises grouping the cells by gene expression profile.

8. The method of claim 1 wherein performing a single cell RNA sequencing on the plurality of differentiated cells to identify genes relating to cellular differentiation further comprises clustering the cell cultures using UMAP or t-SNE; and classifying the cell cultures into a plurality of subtypes based on a primary regulatory gene.

9. The method of claim 8 further comprising determining a plurality of cell types formed.

10. The method of claim 9 further comprising determining the primary regulatory gene found in each of the plurality of cell types.

11. The method of claim 1 wherein the one or more tagged regulatory genes comprise a gene found in a human genome.

12. The method of claim 11 wherein the one or more genes are selected from a group consisting of coding and non-coding genes.

13. A method for identifying a regulatory gene relating to cellular differentiation, the method comprising:
transfecting a plurality of stem cells within a cell culturing system with a test gene;
incubating the cell culturing system under conditions suitable to allow the plurality of stem cells comprising the test gene to differentiate into a plurality of differentiated cells; and
performing single cell RNA sequencing on the plurality of differentiated cells, wherein the single cell RNA sequencing of the plurality of differentiated cells is indicative of a test gene efficacy as a regulatory gene for cellular differentiation.

14. The method of claim 13 wherein the test gene is a gene from a human genome.

15. The method of claim 13 wherein further comprising:
tagging the test gene; and
delivering the test gene to the plurality of stem cells via a Retrovirus.

16. A non-transitory computer readable medium having instructions stored thereon that, when executed, causes an apparatus to perform a method, including: contacting a plurality of stem cells with one or more tagged regulatory genes and a selection marker to form a first plurality of transfected/transduced stem cells; selecting the first plurality of transfected/transduced stem cells; culturing the first plurality of transfected/transduced stem cells under conditions suitable to allow the plurality of transfected/transduced stem cells to differentiate into a plurality of differentiated cells expressing the one or more tagged regulatory genes; and performing a single cell RNA sequencing on the plurality of differentiated cells to identify genes relating to cellular differentiation.

17. An expression vector, comprising:
a coding target gene for RNA sequencing, wherein the coding target gene comprises an untranslated leader sequence or an untranslated trailer sequence; and
a 6 base-pair barcode attached to the untranslated leader sequence or the untranslated trailer sequence.

18. The expression vector of claim 17, wherein the coding target gene comprises only an untranslated trailer sequence, and the 6 base-pair barcode is attached to the untranslated trailer sequence.

19. The expression vector of claim 17, wherein the coding target gene comprises only an untranslated leader sequence, and the 6 base-pair barcode is attached to the untranslated leader sequence.

20. A host cell, comprising: the expression vector of claim 17.
